(19) United States
(12) Patent Application Publication
(10) Pub. No.: US 2025/0290140 A1
(43) Pub. Date: Sep. 18, 2025

(54) TRANSCRIPTOMIC PROFILING

(71) Applicant: The Trustees of Columbia University in the City of New York, New York, NY (US)

(72) Inventors: Harris Wang, New York, NY (US); Yiming Huang, New York, NY (US)

(21) Appl. No.: 18/859,801

(22) PCT Filed: Apr. 28, 2023
(86) PCT No.: PCT/US23/66386

§ 371 Date: Oct. 24, 2024

Related U.S. Application Data
(60) Provisional application No. 63/336,697, filed on Apr. 29, 2022.

(51) Int. Cl. C12Q 1/6883; C12Q 1/6886; C12Q 1/689

(52) U.S. Cl. CPC C12Q 1/6883; C12Q 1/6886; C12Q 1/689; C12Q 2600/118; C12Q 2600/158

(57) ABSTRACT

The present disclosure relates to methods and systems for transcriptomic profiling of a biological sample and use of the transcriptomic profile for disease monitoring, responses to perturbations, and personalized therapies. In particular, the disclosure is related to methods and systems for transcriptomic profiling from host cells (e.g., small and large intestine exfoliated cells) in feces.

### CROSS-REFERENCE TO RELATED APPLICATIONS

This application claims the benefit of U.S. Provisional Application No. 63/336,697, filed Apr. 29, 2022, the content of which is herein incorporated by reference in its entirety.

### STATEMENT REGARDING FEDERALLY—SPONSORED RESEARCH OR DEVELOPMENT

This invention was made with government support under AI132403 and DK118044 awarded by the National Institutes of Health and HR00111920009 awarded by U.S. Department of Defense/DARPA. The government has certain rights in the invention.

### SEQUENCE LISTING

The contents of the electronic sequence listing titled “COLUM-40850.601.xml” (Size: 1,982,275 bytes; and Date of Creation: Apr. 28, 2023) is herein incorporated by reference in its entirety.

### TECHNICAL FIELD

The present disclosure relates to methods and systems for transcriptomic profiling of a biological sample and use of the transcriptomic profile for disease monitoring, responses to perturbations, and personalized therapies. In particular, the disclosure is related to methods and systems for transcriptomic profiling from host cells (e.g., small and large intestine exfoliated cells) in feces.

### BACKGROUND

Inflammatory Bowel Disease (IBD) is a broad term that describes conditions characterized by chronic inflammation of the gastrointestinal tract. The two most common inflammatory bowel diseases are Crohn's disease and ulcerative colitis. IBD is a chronic condition with symptoms that tend to wax and wane with frequent exacerbations. Adequate monitoring is crucial for identifying disease relapse and administering timely treatments. Besides IBD, other chronic colon diseases, such as irritable bowel syndrome, similarly require long-term monitoring and management. Current gut disease management approaches include colonoscopy, stool clinical marker tests, blood tests, and a data-driven IBD tracker. Colonoscopy is the gold standard in monitoring approaches but lacks temporal resolution and is invasive and expensive. Stool clinical marker tests and blood tests are non-invasive but suffer from low resolution or insufficient information for correlation to disease states, respectively. Data-driven IBD trackers are convenient but the data is limited to existing databases due to insufficient information. Thus, non-invasive, cost-effective, and reliable methods and systems are needed to manage chronic diseases.

### SUMMARY

Provided herein are methods and systems for transcriptomic profiling of a biological sample. In some embodiments, the biological sample is a fecal sample. The methods combine amplification (e.g., PCR amplification) of genes of interest with high-throughput sequencing read-outs.

In some embodiments, the methods comprise amplifying one or more target RNA sequences from a sample comprising RNA extracted from a fecal sample from a subject to produce amplicons; and sequencing the amplicons. In some embodiments, the amplicons are single stranded, double stranded, or a combination thereof. In some embodiments, the amplicons are less than about 500 bases in length.

In some embodiments, the RNA extracted from the fecal sample comprises RNA derived from subject cells and RNA derived from gut bacteria. In some embodiments, the one or more target RNA sequences are derived from one or more subject genes. In some embodiments, the one or more subject genes comprise a housekeeping gene, a tissue-specific gene, a cell type-specific gene, a disease related gene, a cell-signaling gene, or combinations thereof. In some embodiments, the methods further comprise determining gene expression for the one or more subject genes.

In some embodiments, the one or more target RNA sequences are about 300 to about 400 nucleotides in length.

In some embodiments, the amplicons are greater than about 150 bases in length. In some embodiments, the amplicons are about 350 to about 500 bases in length.

In some embodiments, the methods further comprise purifying the amplicon based on size prior to sequencing.

In some embodiments, the amplifying comprises contacting the sample with a reverse transcriptase and random hexamer primers under conditions for DNA synthesis to form an cDNA mixture and contacting the cDNA mixture with a DNA polymerase and a pair of oligonucleotide primers configured to specifically amplify each of the one or more target sequences under conditions for amplicon production.

In some embodiments, amplicon production comprises limited cycle PCR amplification. In some embodiments, the limited cycle PCR amplification comprises 5 to 20 amplification cycles.

In some embodiments, the oligonucleotide primers are 20-30 nucleotides in length. In some embodiments, the oligonucleotide primers have a melting temperature of about 62° C. to about 68° C.

In some embodiments, each of the oligonucleotide primers comprises an amplicon identifier sequence. In some embodiments, each amplicon comprises two amplicon identifier sequences flanking a target sequence.

In some embodiments, the amplifying further comprises removing residual RNA from the cDNA mixture. In some embodiments, the methods further comprise removing single stranded nucleic acid impurities from the amplicons.

In some embodiments, the sample further comprises an external RNA control. In some embodiments, the methods further comprise amplifying and sequencing control sequences derived from the external RNA control.

In some embodiments, the methods further comprise profiling the gut microbiome.

In some embodiments, the subject is human. In some embodiments, the subject has or is suspected of having a disease or disorder. In some embodiments, the disease or disorder is a gastrointestinal disease or disorder. In some embodiments, the gastrointestinal disease or disorder is selected from irritable bowel syndrome (IBS), inflammatory bowel diseases (IBD), Crohn's disease (CD), Celiac's disease (CeD), and ulcerative colitis (UC).

Also provided herein are methods for diagnosing a disease or disorder in a subject. The methods comprise generating a transcriptome profile of subject cells in a fecal sample from the subject by a method disclosed herein and comparing the transcriptome profile to a healthy control to determine whether the individual has or has an increased likelihood of having the disease or disorder.

Further provided are methods for monitoring the progression or regression of a disease or disorder in a subject. The comprise acquiring two or more fecal samples from the subject, wherein the two or more fecal samples are separated by a period of time, generating a transcriptome profile of subject cells in the two or more fecal samples by a method disclosed herein, and determining changes in the transcriptome profile between any of the fecal samples. In some embodiments, the methods comprise associating changes in the transcriptome profile with progression or regression of the disease or disorder.

In some embodiments, the disease or disorder is a gastrointestinal disease or disorder. In some embodiments, the gastrointestinal disease or disorder is selected from irritable bowel syndrome (IBS), inflammatory bowel diseases (IBD), Crohn's disease (CD), Celiac's disease (CeD), ulcerative colitis (UC), and colon cancer.

Also provided are methods for evaluating gut health in a subject. In some embodiments, the methods comprise generating a transcriptome profile of subject cells in a first fecal sample from the subject by a method disclosed herein; and comparing the transcriptome profile of the first fecal sample to one or more controls to determine measure of overall gut health.

The methods may further comprise acquiring one or more additional fecal samples from the subject, wherein the one or more additional fecal samples are separated from the first fecal sample or each other by a period of time and generating a transcriptome profile of the one or more additional fecal samples. In some embodiments, the methods comprise identifying changes in the transcriptome profile between any of the fecal samples; and associating changes in the transcriptome profile with changes in gut health.

In some embodiments, the methods comprise generating a transcriptome profile of subject cells in one or more fecal samples from the subject by a method disclosed herein; and comparing the transcriptome profile of the one or more fecal samples to one or more controls to determine measure of overall gut health. In some embodiments, the methods further comprise identifying changes in the transcriptome profile between any of the one or more fecal samples; and associating changes in the transcriptome profile with changes in gut health. In some embodiments, the methods further comprise providing an assessment of gut health.

In some embodiments, the subject is a healthy subject. In some embodiments, the subject is not suffering from a gastrointestinal disease or disorder.

The methods may further comprise signal decomposition to determine the heterogeneity and distribution of specific cell types.

The transcriptomic profiling from small and large intestine exfoliated cells from the fecal sample allows a non-invasive means to prove the transcriptome of the intestines and characterize and diagnose disorders of the gut, including for example, inflammatory bowel disease (IBD) and colitis and chronic diseases, such as, metabolic conditions, and neurological, cardiovascular, and respiratory illnesses, which are associated with changes in gut cells.

The transcriptomic profiling may include any or all of: 16 housekeeping genes (e.g., Gapdh, Gnai3, Dazap2, Tfe3, Sdhd, Trappc 10, Rtca, Dlat, Xpo6, Ndufa9, Ddt, Gpr107, Narf, Tbrg4, Brat1), 50 tissue-specific genes (e.g., from large intestine, small intestine, and brain), 63 cell-type marker genes identified from mice gut single-cell RNA-seq, 126 IBD- and colitis-related genes, and 102 genes identified from colon/cecum RNA-seq.

Other aspects and embodiments of the disclosure will be apparent in light of the following detailed description.

### BRIEF DESCRIPTION OF THE DRAWINGS

FIG. 1 is a schematic of an exemplary exfoliome sequencing method by multiplex PCR based amplicon generation (Exfo-seq).

FIG. 2 is schematic of an exemplary workflow of an amplicon-based exfoliome sequence method. The multiplex PCR reaction setup consists of three key parts (1) primer design for gene targets amplification; (2) multiplex PCR reaction parameters (3) unused primers and undesired product removal. Additionally, a “unique amplicon identifier” (UAI) is introduced on amplification primers to eliminate all bias on amplicon quantification in downstream Illumina library preparation and sequencing. For a given set of genes of interest, criteria used for primers design, parameters involved in multiplex PCR reaction, as well as steps/procedures utilized to remove undesired material and purify gene amplicons are outlined. The resulting gene amplicons are subjected to Illumina library preparation and sequencing for exfoliome RNA profiling.

FIG. 3 shows Exfo-seq can robustly capture gene signals with limited input amounts. Purified human RNA was mixed with E. coli RNA at different ratios and profiled with Exfo-seq. Initial primer sets for the spike-in experiment include 34 amplicon targets on 19 randomly selected genes. Remarkably, host RNA as low as 0.01 ng (0.01% of total RNA) could be robustly amplified and sequenced. Based on a theoretical calculation of amount of RNA extractable from stool, this result suggested that Exfo-seq can be applied on mouse and human stool samples.

FIG. 4 shows the technical and biological reproducibility of Exfo-seq. Exfoliome RNA sequencing was performed twice on individual stool samples (bottom left panel) or samples collected from different mice housed together in the same cage (bottom right panel).

FIG. 5 shows that exfoliome gene expression captured by Exfo-seq is consistent with input and colon tissue as determined by existing standard methods. Exfoliome RNA sequencing on stool samples with external RNA control (ERCC) as spike-in control was compared the quantification of ERCC based on the input concentration (left panel). Exfoliome RNA sequencing on stool samples of mouse fecal RNA abundance was compared to the colon tissue gene expression by conventional RNA-seq (right panel).

FIG. 6 shows Exfo-seq captures gene expression of gut cells from large intestine. Fecal gene expression quantified Exfo-seq was compared to gene expression in different mouse tissues along the gastrointestinal tract determined by conventional RNA-seq. Exfoliome RNA predominantly represented large intestine signals while some small intestine signals were also observed.

FIGS. 7A-7C show Exfo-seq captured increased cell exfoliation and inflammation trajectory in mouse DSS-induced colitis model. FIG. 7A is a schematic of the experimental design using a DSS induced mouse colitis model. FIG. 7B is a graph of the increase of cell/RNA exfoliation for mouse with colitis. FIG. 7C is a graph showing detection of development trajectory of DSS-induced colitis.

FIGS. 8A-8C show Exfo-seq captured temporal differential gene expression in mouse DSS-induced colitis model. Analysis of the RNA exfoliome data from the DSS-induced mouse colitis model showed longitudinal differential gene expression of mouse gastrointestinal tract (FIG. 8A) enabling identification of early-responding biomarkers (FIG. 8B). Further analysis of these differentially expressed genes showed their longitudinal expression (FIG. 8C) in DSS-induced colitis model.

FIGS. 9A and 9B show Exfo-seq captured kinetics of cell type changes by signal decomposition in mouse DSS-induced colitis model. Using a previously established computation frameworks with RNA data generated by Exfo-seq, the cell-type composition of exfoliated cells RNA was determined (FIG. 9A). FIG. 9B shows the analysis used on exfoliome data from the DSS-induced mouse colitis model which identified longitudinal cell-type composition changes, e.g., expansion of specific immune cell types.

FIGS. 10A-10C show Exfo-seq captured temporal dynamics of mouse gut cell gene expression in a non-perturbated mouse model. FIG. 10A a schematic of the experimental design to apply Exfo-seq to an un-perturbed mouse model to monitor gut gene expression fluctuation for 6 weeks. FIGS. 10B and 10C show that housekeeping genes generally fluctuated less in comparison to inflammation-related genes.

FIGS. 11A-11C show combining Exfo-seq and rRNA 16S-seq captured temporal host-microbe interaction in a non-perturbated mouse model. Exfoliome RNA data was combined with gut microbiota profiling by conventional 16S rRNA sequencing in the un-perturbed mouse model. FIG. 11A shows the global shift in the gut microbiota profile over time, which may explain the variation of some host gene expression seen in FIG. 10. FIGS. 11B and 11C show correlation and links between microbiota species and gene expression of gastrointestinal.

FIG. 12 is graphs showing that Exfo-seq demonstrates higher sensitivity in quantifying biomarkers. Exfo-seq exfoliome RNA quantification from a C. rodentium infection mouse mild colitis model (right) was compared to an ELISA assay (left) on a well-known inflammation biomarker Len2 to quantify its protein level (Lipocalin) in stool with a commercial kit.

FIG. 13 shows Exfo-seq robustly quantified exfoliome of human stool sample collected 5 years ago with high technical reproducibility.

FIG. 14 shows Exfo-seq captured temporal exfoliome fluctuations within individuals and variations between individuals in a healthy cohort. Exfoliome RNA sequencing on human stool samples from either the same healthy donors at different time points or different healthy donors identified the temporal gut gene expression fluctuation within individuals and variation between individuals.

FIG. 15 shows Exfo-seq separated IBS patients from healthy individuals and identified IBS gene signatures. Stool exfoliome RNA sequencing was performed on samples collected from active IBS patients and their exfoliome profile was compared to samples from healthy individuals. Exfoliome RNA of IBS patients were distinct from healthy individuals, and analysis of detailed gene-level differences identified a set of genes that were highly expressed in active IBS patients, which could imply disease etiologies or be used as biomarkers for IBS.

### DETAILED DESCRIPTION

The disclosed systems, compositions, and methods advance methods transcriptomic profiling of a biological sample, particularly fecal samples.

Greater than twenty percent of gut epithelial cells are shed each day according to previous reports. These cells and their nucleic acids material (e.g., exfoliome RNA) can be found in stool and since they originated from the gastrointestinal tract are ideal material to use for gathering information of overall gut health. However, extremely low signals are captured by existing methods due to extremely low amounts and quality of host cells in fecal samples and high contamination from microbial sources. Additionally, the rapid degradation of RNA results in poor quantity of RNA of a quality suitable for use. The majority (greater than 99%) of cells in fecal matter are due to the trillions of gut microbes that reside in the gastrointestinal tract. Thus, although there are exfoliated host cells and host nucleic acids in stool, it is challenging to capture these signals and quantify them. Previous attempts to profile these exfoliated nucleic acids using standard ploy-A capture method generate a very low ratio of usable signals, which is not sufficient for robust quantification.

Disclosed herein are methods for transcriptomic profiling with improved efficiency, accuracy, and consistency over existing methods. The disclosed methods overcome limitations of RNA fragility, low input RNA concentration, and high background contamination commonly associated with complex samples, such as fecal samples. In some embodiments, the methods include multiplex PCR to amplify gene signals of interests combined with next-generation sequencing (NGS). The disclosed methods can capture gene signatures from 0.01 ng of human RNA (less than 20 cells or 0.01% of total RNA) with high contamination (>99.99%). The disclosed methods further facilitate monitoring and management of chronic diseases, such as gastrointestinal diseases and disorders, in a non-invasive, convenient, sensitive, and cost-effective way. Furthermore, the disclosed methods can be designed to probe for specific gene signatures for evaluating patient health and optimizing therapy.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule, for example, as in polymerase chain reaction (PCR).

The term “amplicon” or “amplified product” refers to a segment of nucleic acid, generally DNA, generated by an amplification process such as the PCR process.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural, and other sequences). The sequences that are located in the S′ of the coding regions and that are present on the mRNA are referred to as 5′ non-translated or untranslated sequences. The sequences that are located 3′ or downstream of the coding region and that are present on the mRNA are referred to as 3′ non-translated or 3′ untranslated sequences.

The terms “primer,” “primer sequence,” “primer oligonucleotide,” and “amplification oligonucleotide” as used herein, refer to an oligonucleotide, whether naturally occurring or synthetic, which is capable of acting as a point of initiation of synthesis of an extension product that is a complementary strand of nucleic acid (all types of DNA or RNA) when placed under suitable amplification conditions (e.g., buffer, salt, temperature and pH) in the presence of nucleotides and an agent for nucleic acid polymerization (e.g., a DNA-dependent or RNA-dependent polymerase). The primers of the present disclosure can be of any suitable size, and desirably comprise, consist essentially of, or consist of about 15 to 50 nucleotides.

As used herein, the terms “primer set,” “set,” or “set of primers” refer to two or more oligonucleotides which together are capable of priming the amplification of a target sequence. In certain embodiments, the term “primer set” refers to a pair of oligonucleotides including a first oligonucleotide that hybridizes with the 5′-end of the target sequence or target nucleic acid to be amplified and a second oligonucleotide that hybridizes with the complement of the target sequence or target nucleic acid to be amplified at the 3′ end.

The primers may be modified in any suitable manner so as to stabilize or enhance the binding affinity of the oligonucleotide for its target. For example, an oligonucleotide sequence as described herein may comprise one or more modified oligonucleotide. Modified nucleotides are nucleotides or nucleotide triphosphates that differ in composition and/or structure from natural nucleotides and nucleotide triphosphates. Modifications include those naturally occurring that result from modification by enzymes that modify nucleotides, such as methyltransferases. Modified nucleotides also include synthetic or non-naturally occurring nucleotides. For example, modified nucleotides include those with 2′ modifications, such as 2′-O-methyl and 2′-fluoro. Other 2′-modified nucleotides are known in the art and are described in, for example U.S. Pat. No. 9,096,897, which is incorporated herein by reference in its entirety. Modified nucleotides or nucleotide triphosphates used herein may, for example, be modified in such a way that, when the modifications are present on one strand of a double-stranded nucleic acid where there is a restriction endonuclease recognition site, the modified nucleotide or nucleotide triphosphates protect the modified strand against cleavage by restriction enzymes.

The terms “target sequence” and “target nucleic acid (e.g., RNA) sequence” are used interchangeably herein and refer to a specific nucleic acid sequence, the presence, absence, or level of which is to be analyzed by the disclosed method. In the context of the present disclosure, a target sequence preferably includes a nucleic acid sequence to which one or more oligonucleotides will hybridize and from which amplification will initiate.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human). Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment of the methods provided herein, the subject is a human.

The term “contacting” as used herein refers to bring or put in contact, to be in or come into contact. The term “contact” as used herein refers to a state or condition of touching or of immediate or local proximity.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Transcriptomic Profiling

In a broad sense, transcriptomic profiling is analysis of a set of RNA molecules expressed in some given sample, such as a particular cell or group of cells, tissues, organism. Transcriptome profiling is currently performed using hybridization or sequencing-based methodologies. However, when used with complex samples, samples which have low RNA amounts, or samples which have large amounts of contaminating nucleic acids, particularly from other RNA sources, these current methods suffer from limitations such as low resolution, quantification, specificity, and/or sensitivity. The methods disclosed herein overcome those limitations, particularly for fecal samples, with increased scalability (e.g., monitor hundreds to thousands of genes in a single reaction) and lower cost.

In some embodiments, the methods comprise amplifying one or more target RNA sequences from a sample comprising RNA extracted from a subject fecal sample to produce amplicons of less than about 500 bases in length and sequencing the amplicons.

In some embodiments, the fecal samples are freshly collected samples. Additionally, under certain conditions, fresh fecal samples are not analyzed immediately and are instantly frozen at −80° C. to maintain integrity. However, the fecal samples do not have to be freshly collected. Thus, samples collected 1, 2, 3, 4, 5, 6 or more years ago may be employed. The historical samples may have been frozen, at a suitable temperature, such as −80° C. for example, for storage. Lyophilized fecal samples may also be suitable for use with the disclosed methods. The sample may be frozen with or without the addition of stabilizing agents. When ready for use, frozen or lyophilized samples may be thawed in the presence or absence of additional stabilizing agents (e.g., a stabilization buffer).

In general, stabilizing agents, for example as in a stabilizing buffer, are those chemical agents which maintain an appropriate pH, as well as the use of chelating agents to prevent the phenomenon of metal redox cycling or the binding of metal ions to the phosphate backbone of nucleic acids. The term “chelator” or “chelating agent” as used herein will be understood to mean a chemical that will form a soluble, stable complex with certain metal ions (e.g., Ca²⁺ and Mg²⁺), sequestering the ions so that they cannot normally react with other components, such as deoxyribonucleases (DNase) or endonucleases (e.g. type I, II and III restriction endonucleases) and exonucleases (e.g. 3′ to 5′ exonuclease), enzymes which are abundant in the GI tract.

Only a portion of the collected fecal sample may need to be employed in the methods of the invention to achieve reliable results. A fecal sample of less than 1 gram may allow multiple rounds of the methods disclosed herein. Thus, reliable results may utilize less than 1 gram total of a fecal sample. In some embodiments, the fecal sample employed in the methods disclosed herein is less than about 1 g, less than about 0.75 g, less than 0.5 g, less than 0.25 g, less than 0.1 g, less than 0.05 g, or less.

The fecal sample may be processed in an appropriate volume of homogenization buffer to facilitate RNA extraction. Homogenization of stool can be performed manually, or through the use of additional mechanical agitation methods. In some embodiments, the homogenization is performed using beads.

In some embodiments, the processing comprises filtering the fecal sample. For example, the fecal sample may be subjected to conditions sufficient to filter the sample using gravitational filtration, centrifugal filtration, filter stacking, sedimentation, passive filtering, or filtration using a mesh, membrane, or other filtration mechanism. A filter may comprise a membrane, beads, diaphragms, colloids, weir filters, pillar filters, cross-flow filters, solvent filters, sieves, or any other filter.

In some embodiments, the processing comprises lysis of one or more cells or cell types in the fecal sample. In some embodiments, the lysis is performed using one or more members selected from the group consisting of ultrasonic lysis, mechanical lysis, biological lysis, and chemical lysis. In some embodiments, the lysis is accomplished by the same buffer as used in the homogenization or RNA extraction.

RNA can be extracted and purified using any suitable technique. For example, in some embodiments, RNA can be extracted using TRIzol (Invitrogen, Carlsbad, Calif.) and purified using a variety of RNA preparation kits. RNA can be further purified using DNase treatment to eliminate any contaminating DNA and to eliminate contaminants that interfere with cDNA synthesis (e.g., by precipitation). RNA integrity can be evaluated by running electropherograms, and an RNA integrity number (RIN, a correlative measure that indicates intactness of mRNA) can be determined, if desired. Following RNA extraction, the resulting RNA concentrations can be determined using any suitable method.

A transcriptome profile may refer to all RNA molecules in a cell (including mRNA, rRNA, tRNA and other non-coding RNA products) or a subset of RNA molecules in a cell, such as mRNA molecules. Accordingly, the sample may comprise any or all of the types of RNA molecules, e.g., mRNA, rRNA, tRNA and other non-coding RNA products, or a subset thereof. The RNA used in the methods herein is derived from a fecal sample, thus the extracted RNA includes RNA derived from subject cells found in the fecal sample (e.g., cells exfoliated from various locations all the GI tract or elsewhere in the body) and/or RNA derived from gut bacteria cells.

In some embodiments, the one or more target RNA sequences are derived from one or more subject, or host, genes. Thus, the methods amplify RNA derived from subject cells found in the fecal sample. In some embodiments, the methods profile the RNA from cells exfoliated from various locations in the GI tract, referred to herein as exfoliome RNA. The one or more genes may include, but are not limited to, housekeeping genes, tissue-specific genes, cell type-specific genes, disease-related genes, and/or cell-signaling genes.

In some instances, the one or more target RNA sequences comprises one or more target sequences from genes listed in Tables 1 and 2. In some instances, the one or more target RNA sequences comprises at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, or at least about 50 targets. In some instances, the one or more target RNA sequences comprises at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, or at least about 50 targets from those listed in Tables 1 and 2.

The extracted RNA is reverse transcribed into cDNA using suitable primers. The primers can comprise a portion complementary to a region of the target sequence and/or can comprise non-specific sequences for reverse transcription of the whole transcriptome or a portion thereof. In some embodiments, the primers comprise a portion complementary to a region of the target RNA, such as in a constant region of the target or to a poly-A tail of the mRNA. In some embodiments, the primers include sequence specific, polydT, and/or random hexamer primers. In select embodiments, the primers include random hexamer primers.

In some embodiments, the extracted RNA can be non-specifically transcribed into cDNA which is followed by specific amplification of the target sequences using a DNA polymerase. In some embodiments, the amplification reaction including contacting the sample with a reverse transcriptase and random hexamer primers under conditions for DNA synthesis and then contacting the resulting cDNA with a DNA polymerase and a pair of oligonucleotide primers specific for each of the one or more target sequences under conditions for amplicon production.

Any enzyme having polymerase activity can be used in the amplification, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript® family of enzymes), ThermoScript® family of enzymes, HIV-1, and RAV2 reverse transcriptases.

“Conditions for DNA synthesis” and “conditions for amplicon production,” as used herein, refers to conditions that promote annealing and/or extension of the primers. Such conditions are well-known in the art and depend on the amplification method selected. Amplification conditions encompass all reaction conditions including, but not limited to, temperature and/or temperature cycling, buffer, salt, ionic strength, pH, and the like.

Amplification (e.g., amplicon production and cDNA synthesis) can be performed using any suitable nucleic acid sequence amplification method. In some embodiments, the amplification includes, but is not limited to, polymerase chain reaction (PCR), reverse-transcriptase PCR (RT-PCR), real-time PCR, transcription-mediated amplification (TMA), rolling circle amplification, nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA), and ligase chain reaction (LCR). In some embodiments, the cDNA synthesis and/or amplicon production includes polymerase chain reaction (PCR).

In some embodiments, cDNA generation and/or amplicon production uses limited cycle PCR, for example about 5 to about 25 cycles. Limited cycle PCR amplification is PCR amplification in which the reaction is stopped while in exponential phase such that the target sequence is amplified in a quantitative manner. In some embodiments, amplicon production uses about 10 to about 20 (e.g., 10, 11, 12, 13, 14, 15, 16, 17, 18, 19 or 20) cycles of PCR.

Primers based on the nucleotide sequences of target sequences can be designed for use in amplification of the target sequences. The exact composition of the primer sequences is not critical to the invention, but for most applications the primers hybridize to specific sequences of under stringent conditions, particularly under conditions of high stringency. The primers for a PCR reaction are designed to hybridize to regions in their corresponding template to produce an amplifiable segment. In some embodiments, the primers have a region of hybridization with the target of about 20 to about 30 (e.g., 20, 21, 22, 23, 24, 25, 26, 27, 28, 29, or 30) nucleotides in length.

Different primer pairs can anneal and melt at about the same temperatures (e.g., within 1, 2, 3, 4, 5, 6, 7, 8, 9 or 10° C.). Preferably, the primers are chosen for a melting temperature of about 60° C. to about 60° C. In some embodiments, the primers have a melting temperature of about 62° C. to about 68° C. (e.g., about 62, about 63, about 64, about 65, about 66, about 67, or about 68° C.).

Primers can be designed according to known parameters for avoiding secondary structures and self-hybridization. Algorithms for the selection of primer sequences are generally known, and are available in commercial software packages.

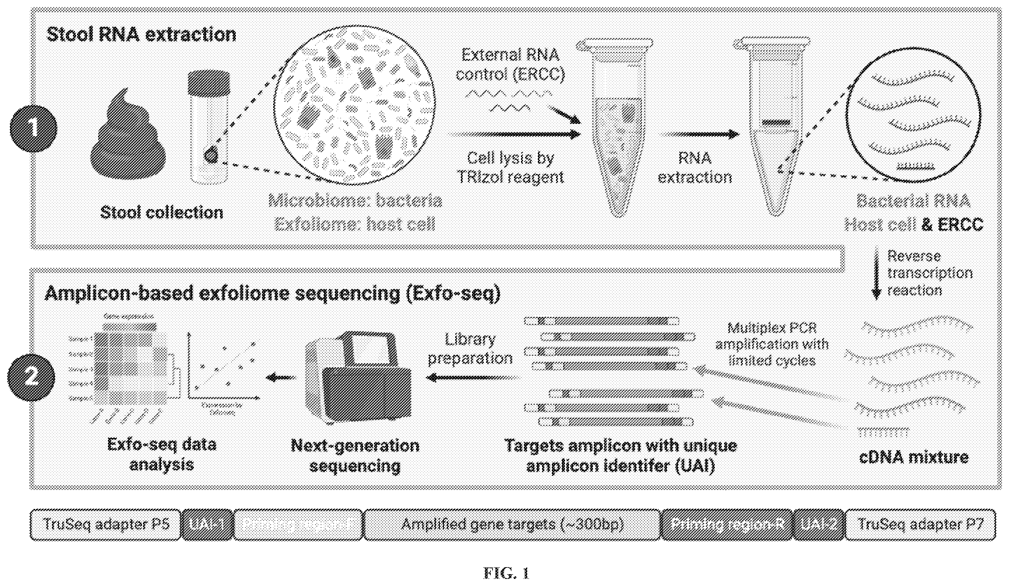

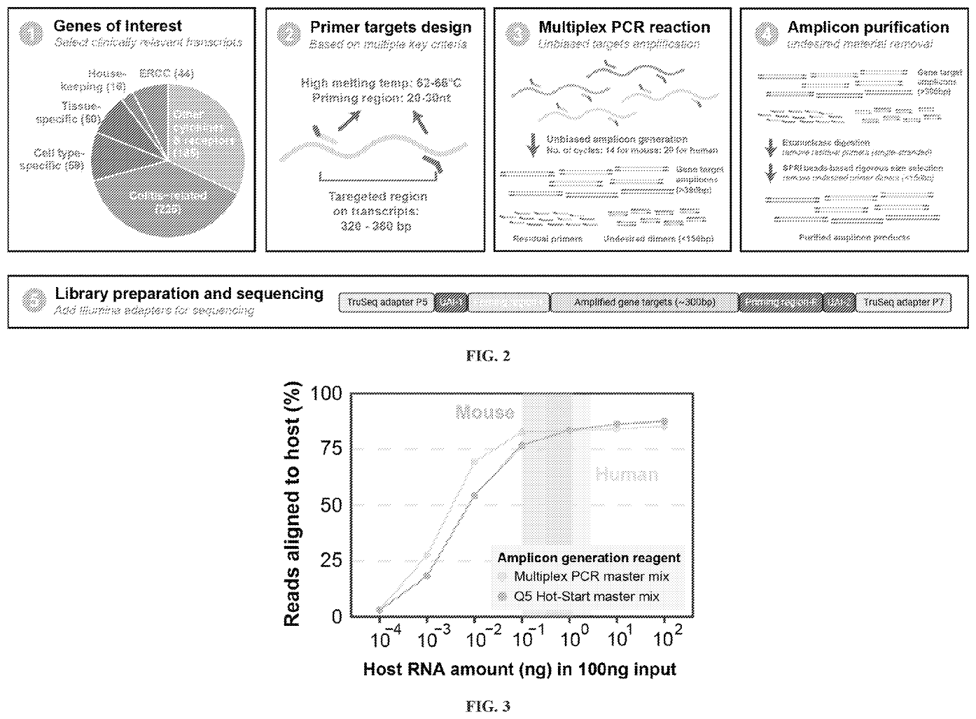

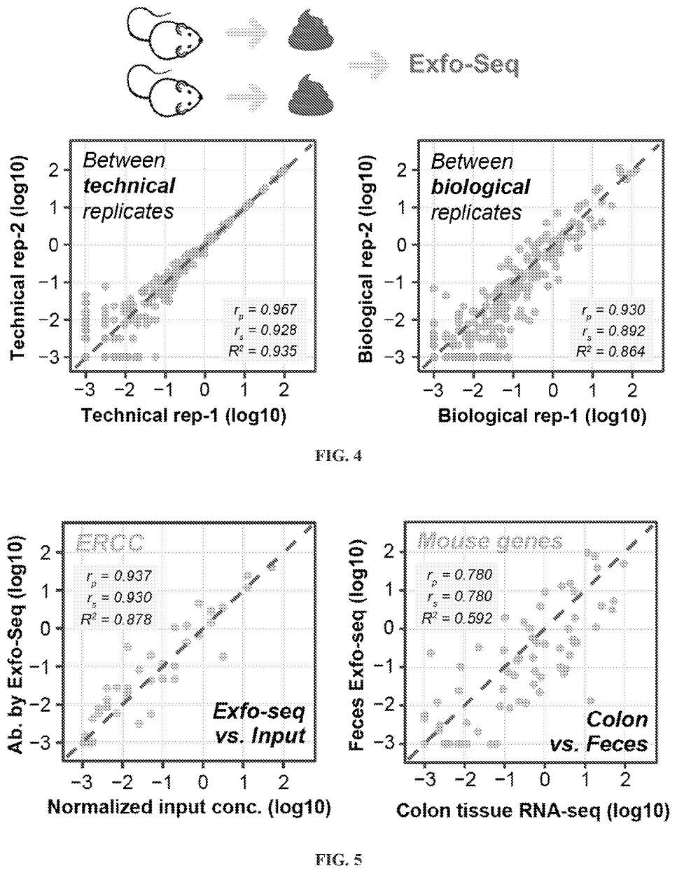

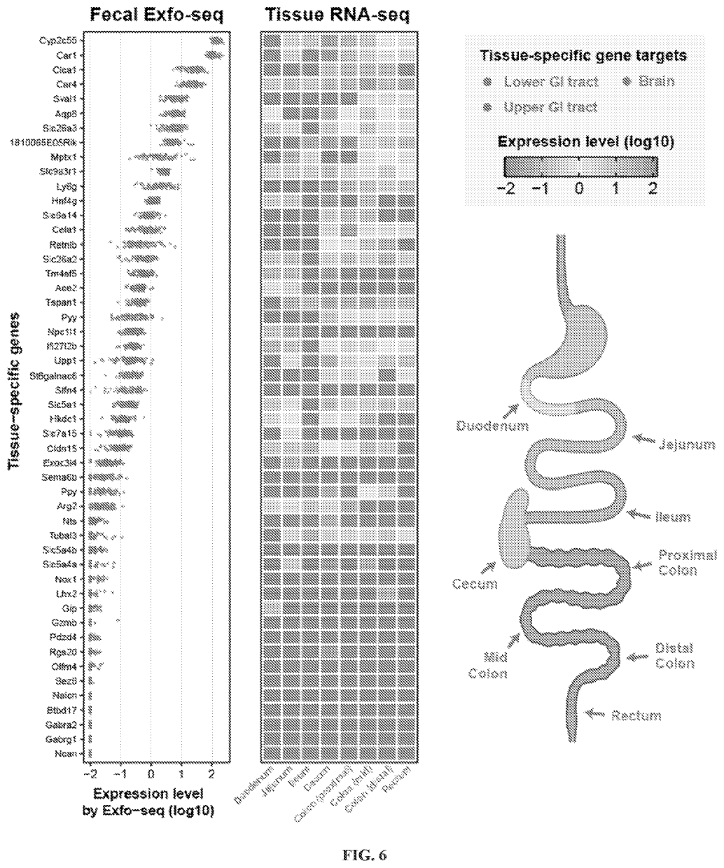

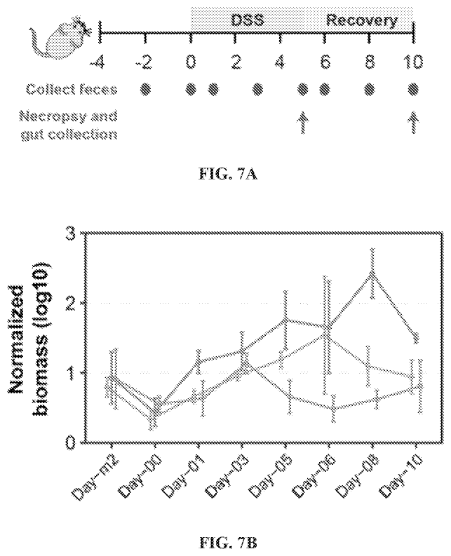

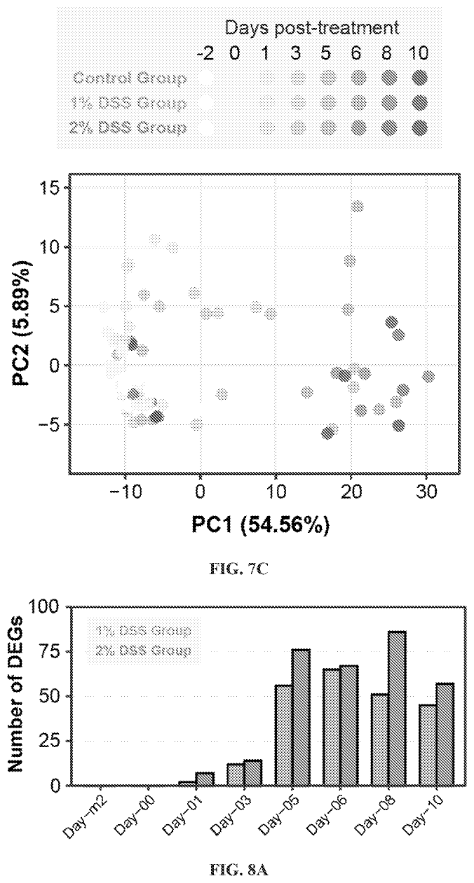

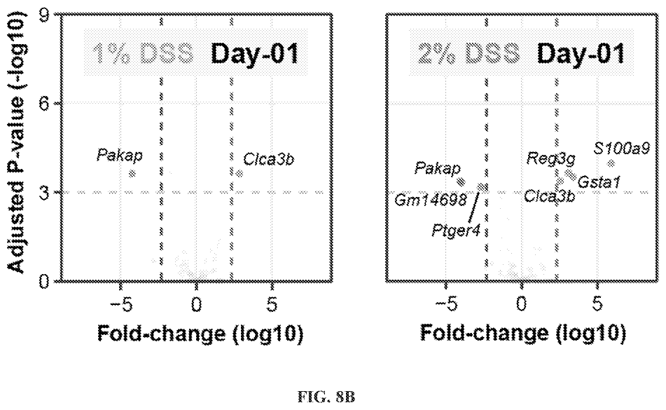

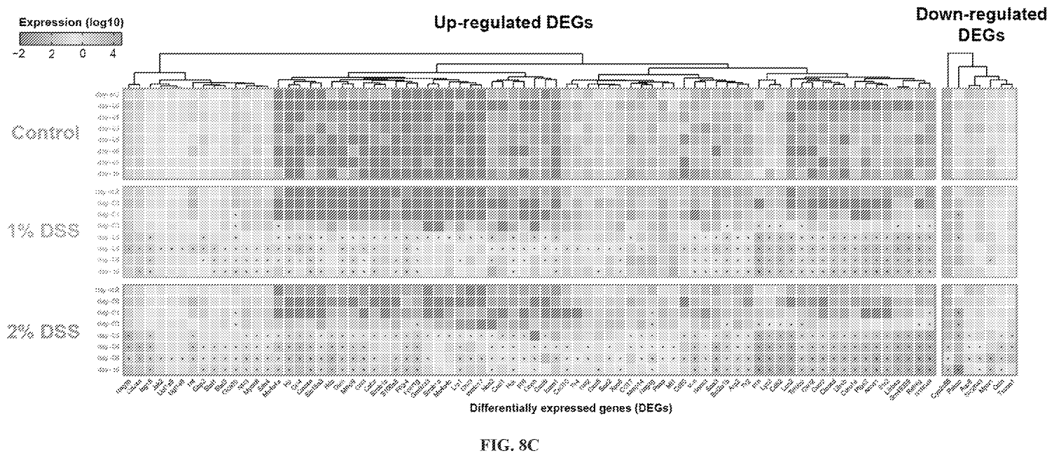

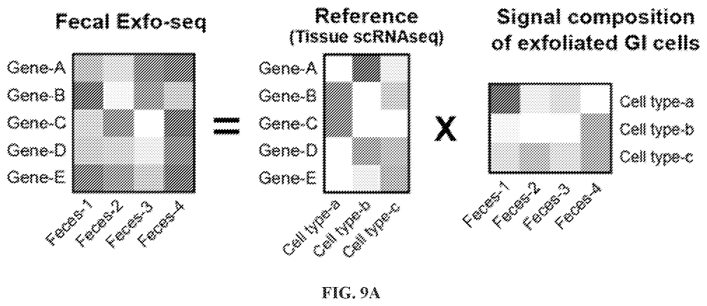

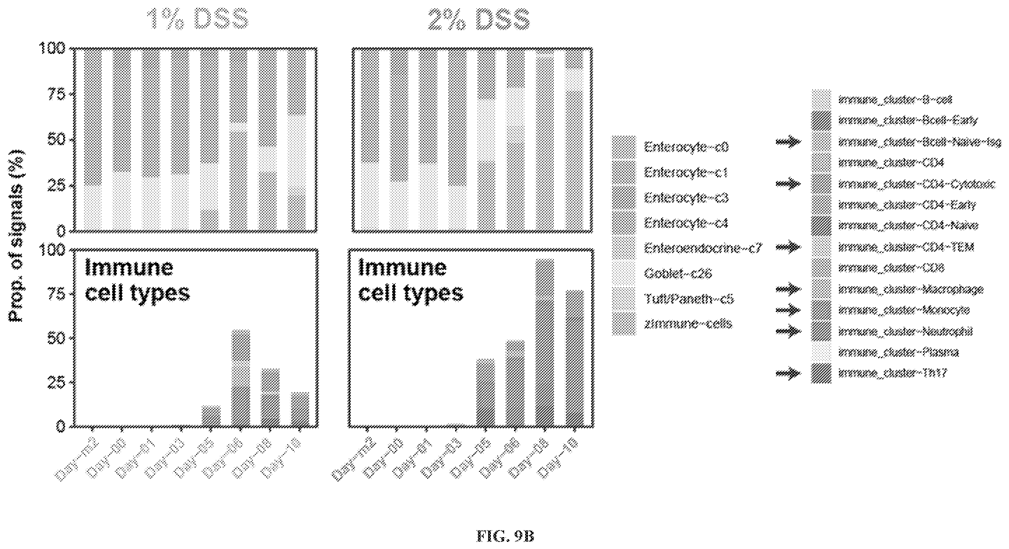

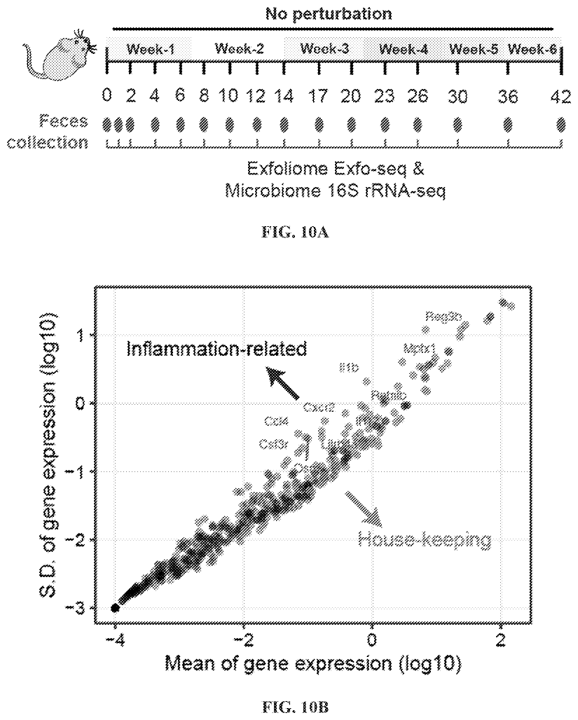

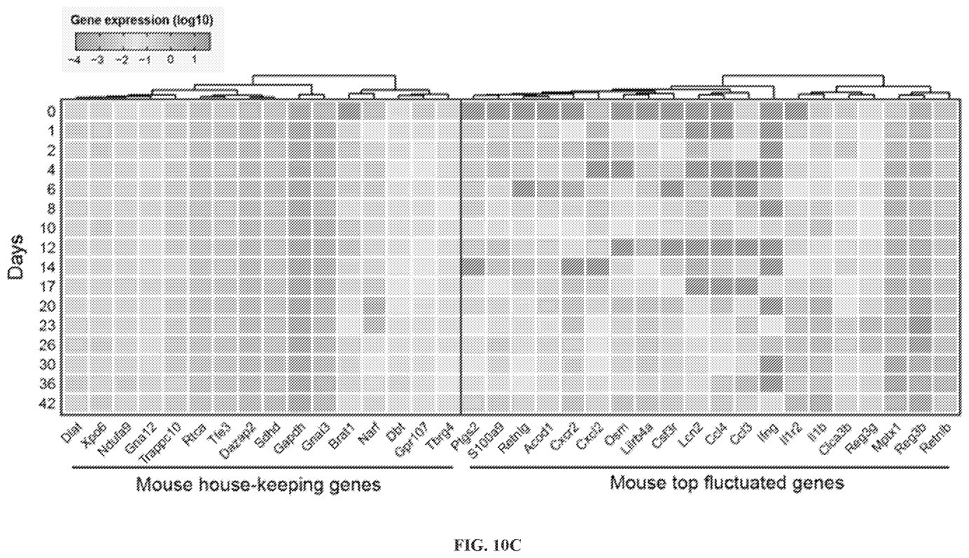

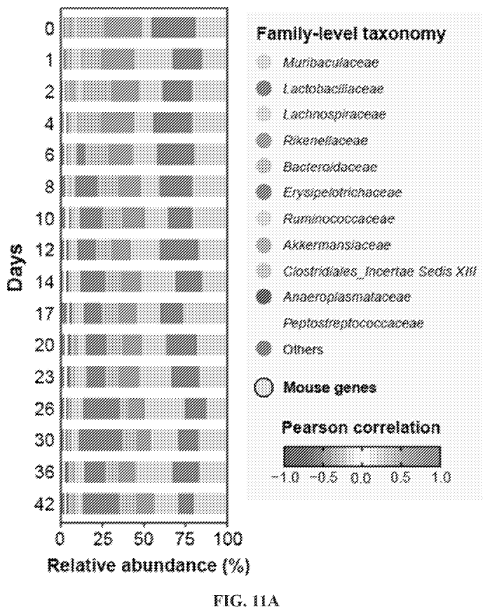

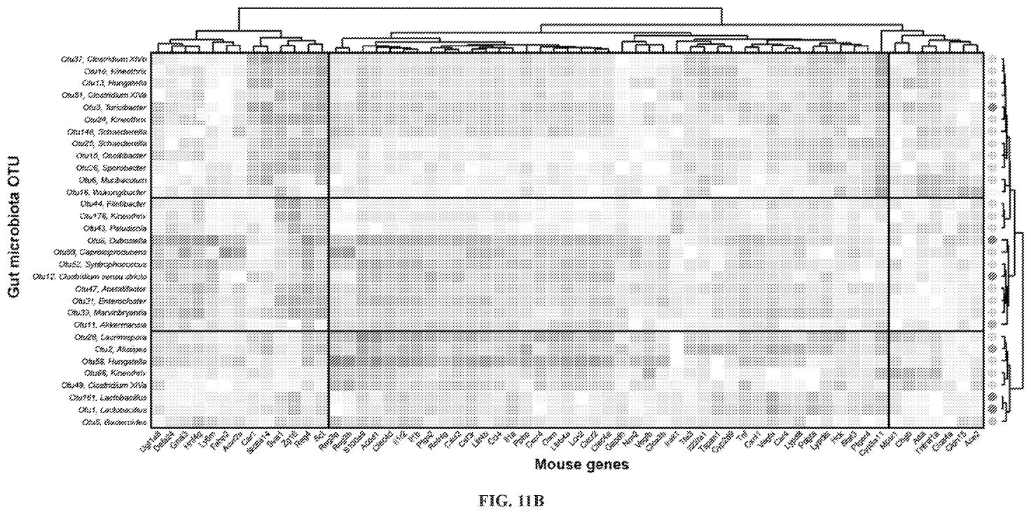

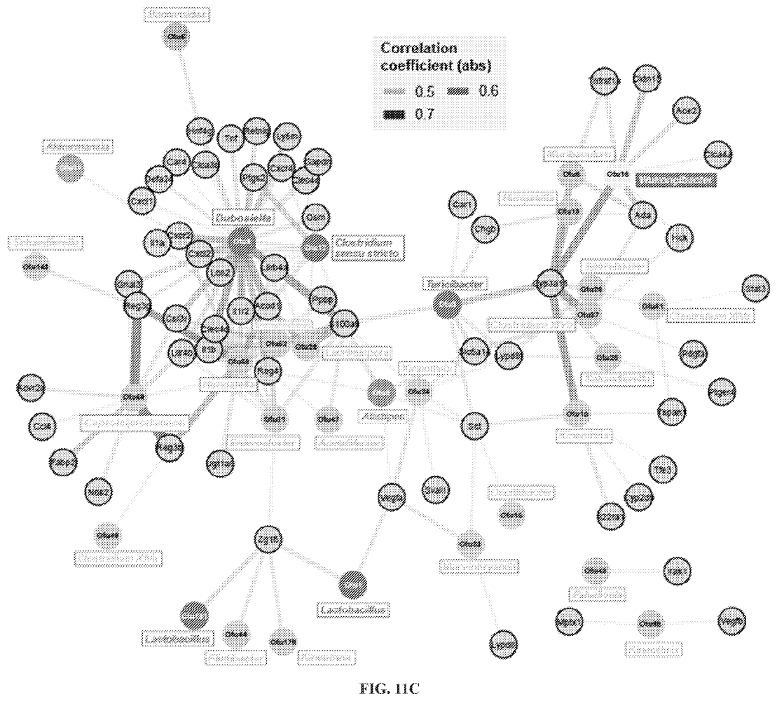

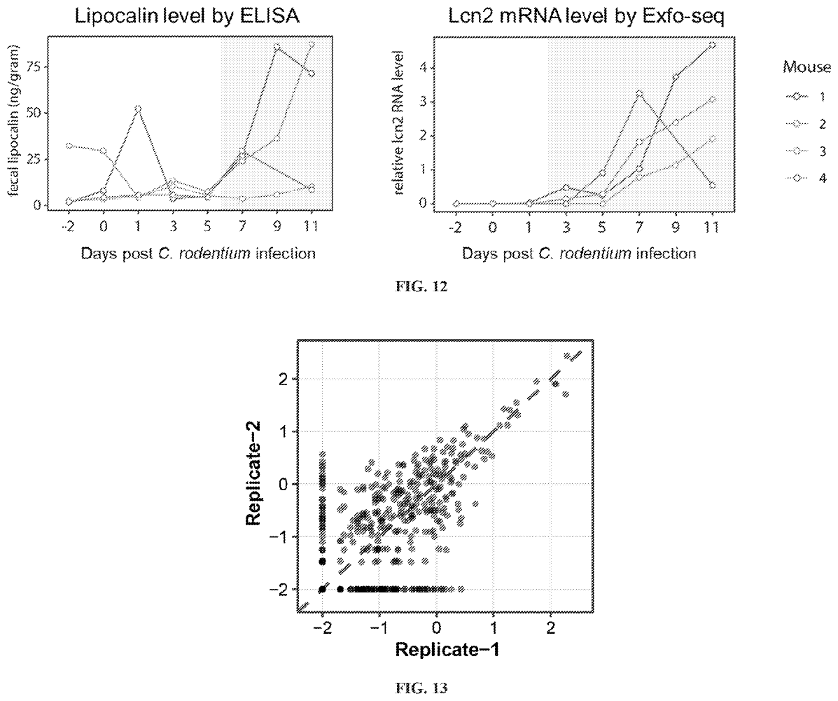

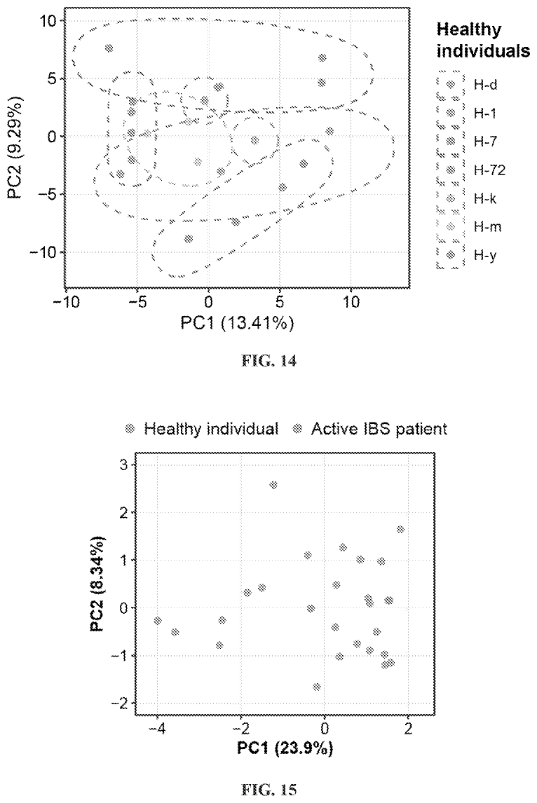

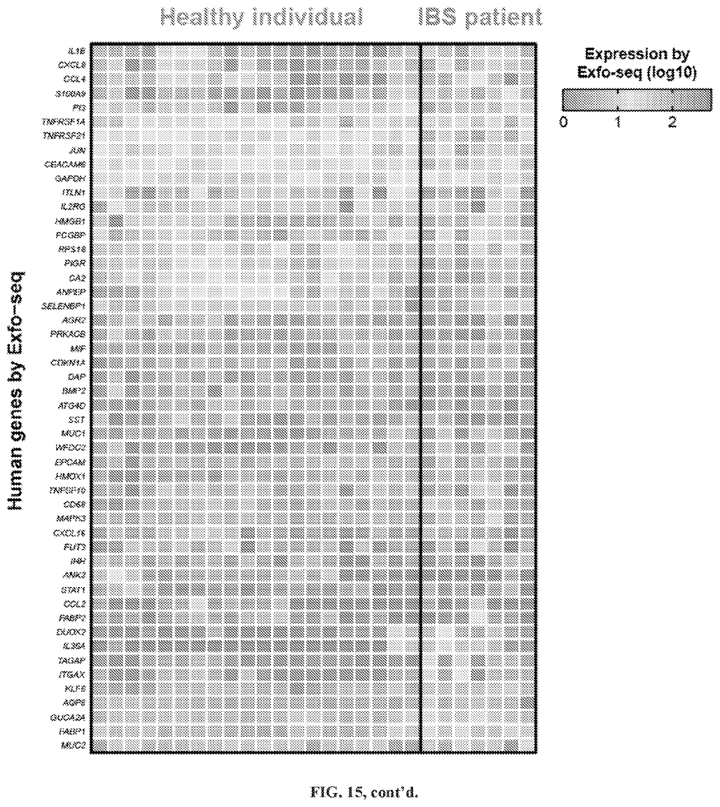

The primers may further comprise an amplicon identifier. An amplicon identifier may include a specific series of nucleotides which do not anneal with the target may be included in each primer sequence, resulting in amplicons which include the target sequence flanked by 5′ and 3′ sequences comprising an amplicon identifier. In some embodiments, the amplicon identifier comprises 4 or more (e.g., 4, 5, 6, 7, 8, 9, 10 or more) consecutive nucleotides of any sequence. As each primer may comprise an amplicon identifier the total resolving power of the identifier is the combination of the two amplicon identifiers. As shown in FIGS. 1 and 2, these unique amplicon identifiers or UAI flank the target sequence and provide a mechanism to eliminate any bias introduced by the library preparation and sequencing, and the addition of any adaptor sequences for using in the downstream sequencing or library preparation, as described below.

The pairs of primers are usually chosen to amplify target sequences of about 300 to about 400 bases in length. In some embodiments, the target sequences are about 300 to about 400 bases in length. The amplicons may be about 300 to about 400 bases, about 310 to about 400 bases, about 320 to about 400 bases, about 330 to about 400 bases, about 340 to about 400 bases, about 350 to about 400 bases, about 360 to about 400 bases, about 370 to about 400 bases, about 380 to about 400 bases, about 390 to about 400 bases, about 300 to about 390 bases, about 310 to about 390 bases, about 320 to about 390 bases, about 330 to about 390 bases, about 340 to about 390 bases, about 350 to about 390 bases, about 360 to about 390 bases, about 370 to about 390 bases, about 380 to about 390 bases, about 300 to about 380 bases, about 310 to about 380 bases, about 320 to about 380 bases, about 330 to about 380 bases, about 340 to about 380 bases, about 350 to about 380 bases, about 360 to about 380 bases, about 370 to about 380 bases, about 300 to about 370 bases, about 310 to about 370 bases, about 320 to about 370 bases, about 330 to about 370 bases, about 340 to about 370 bases, about 350 to about 370 bases, about 360 to about 370 bases, about 300 to about 360 bases, about 310 to about 360 bases, about 320 to about 360 bases, about 330 to about 360 bases, about 340 to about 360 bases, about 350 to about 360 bases, about 300 to about 350 bases, about 310 to about 350 bases, about 320 to about 350 bases, about 330 to about 350 bases, about 340 to about 350 bases, about 300 to about 340 bases, about 310 to about 340 bases, about 320 to about 340 bases, about 330 to about 340 bases, about 300 to about 330 bases, about 310 to about 330 bases, about 320 to about 330 bases, about 300 to about 320 bases, about 310 to about 320 bases, or about 300 to about 310 bases in length.

The pairs of primers are usually chosen so as to generate amplicons of at least about 150 bases/basepairs in length and less than about 500 bases/basepairs in length. The resulting amplicons may be double or single stranded.

In some embodiments, the amplicons are about 150 to about 500 bases/basepairs, about 150 to about 450 bases/basepairs, about 150 to about 400 bases/basepairs, about 150 to about 350 bases/basepairs, about 150 to about 300 bases/basepairs, about 150 to about 250 bases/basepairs, about 150 to about 200 bases/basepairs, about 200 to about 500 bases/basepairs, about 200 to about 450 bases/basepairs, about 200 to about 400 bases/basepairs, about 200 to about 350 bases/basepairs, about 200 to about 300 bases/basepairs, about 200 to about 250 bases/basepairs, about 250 to about 500 bases/basepairs, about 250 to about 450 bases/basepairs, about 250 to about 400 bases/basepairs, about 250 to about 350 bases/basepairs, about 250 to about 300 bases/basepairs, about 300 to about 500 bases/basepairs, about 300 to about 450 bases/basepairs, about 300 to about 400 bases/basepairs, about 300 to about 350 bases/basepairs, about 350 to about 500 bases/basepairs, about 350 to about 450 bases/basepairs, about 350 to about 400 bases/basepairs, about 400 to about 500 bases/basepairs, about 400 to about 450 bases/basepairs, or about 450 to about 500 bases/basepairs in length.

In select embodiments, the amplicons are about 350 to about 500 bases/basepairs in length. The amplicons may be about 350 to about 500 bases/basepairs, about 360 to about 500 bases/basepairs, about 370 to about 500 bases/basepairs, about 380 to about 500 bases/basepairs, about 390 to about 500 bases/basepairs, about 400 to about 500 bases/basepairs, about 410 to about 500 bases/basepairs, about 420 to about 500 bases/basepairs, about 430 to about 500 bases/basepairs, about 440 to about 500 bases/basepairs, about 450 to about 500 bases/basepairs, about 460 to about 500 bases/basepairs, about 470 to about 500 bases/basepairs, about 480 to about 500 bases/basepairs, about 490 to about 500 bases/basepairs, about 350 to about 490 bases/basepairs, about 360 to about 490 bases/basepairs, about 370 to about 490 bases/basepairs, about 380 to about 490 bases/basepairs, about 390 to about 490 bases/basepairs, about 400 to about 490 bases/basepairs, about 410 to about 490 bases/basepairs, about 420 to about 490 bases/basepairs, about 430 to about 490 bases/basepairs, about 440 to about 490 bases/basepairs, about 450 to about 490 bases/basepairs, about 460 to about 490 bases/basepairs, about 470 to about 490 bases/basepairs, about 480 to about 490 bases/basepairs, about 350 to about 480 bases/basepairs, about 360 to about 480 bases/basepairs, about 370 to about 480 bases/basepairs, about 380 to about 480 bases/basepairs, about 390 to about 480 bases/basepairs, about 400 to about 480 bases/basepairs, about 410 to about 3480 bases/basepairs, about 420 to about 480 bases/basepairs, about 430 to about 480 bases/basepairs, about 440 to about 480 bases/basepairs, about 450 to about 480 bases/basepairs, about 460 to about 480 bases/basepairs, about 470 to about 480 bases/basepairs, about 350 to about 470 bases/basepairs, about 360 to about 470 bases/basepairs, about 370 to about 470 bases/basepairs, about 380 to about 470 bases/basepairs, about 390 to about 470 bases/basepairs, about 400 to about 470 bases/basepairs, about 410 to about 470 bases/basepairs, about 420 to about 470 bases/basepairs, about 430 to about 470 bases/basepairs, about 440 to about 470 bases/basepairs, about 450 to about 470 bases/basepairs, about 460 to about 470 bases/basepairs, about 350 to about 460 bases/basepairs, about 360 to about 460 bases/basepairs, about 370 to about 460 bases/basepairs, about 380 to about 460 bases/basepairs, about 390 to about 460 bases/basepairs, about 400 to about 460 bases/basepairs, about 410 to about 460 bases/basepairs, about 420 to about 460 bases/basepairs, about 430 to about 460 bases/basepairs, about 440 to about 460 bases/basepairs, about 450 to about 460 bases/basepairs, about 350 to about 450 bases/basepairs, about 360 to about 450 bases/basepairs, about 370 to about 450 bases/basepairs, about 380 to about 450 bases/basepairs, about 390 to about 450 bases/basepairs, about 400 to about 450 bases/basepairs, about 410 to about 450 bases/basepairs, about 420 to about 450 bases/basepairs, about 430 to about 450 bases/basepairs, about 440 to about 450 bases/basepairs, about 350 to about 440 bases/basepairs, about 360 to about 440 bases/basepairs, about 370 to about 440 bases/basepairs, about 380 to about 440 bases/basepairs, about 390 to about 440 bases/basepairs, about 400 to about 440 bases/basepairs, about 410 to about 440 bases/basepairs, about 420 to about 440 bases/basepairs, about 430 to about 440 bases/basepairs, about 350 to about 430 bases/basepairs, about 360 to about 430 bases/basepairs, about 370 to about 430 bases/basepairs, about 380 to about 430 bases/basepairs, about 390 to about 430 bases/basepairs, about 400 to about 430 bases/basepairs, about 410 to about 430 bases/basepairs, about 420 to about 430 bases/basepairs, about 350 to about 420 bases/basepairs, about 360 to about 420 bases/basepairs, about 370 to about 420 bases/basepairs, about 380 to about 420 bases/basepairs, about 390 to about 420 bases/basepairs, about 400 to about 420 bases/basepairs, about 410 to about 420 bases/basepairs, about 350 to about 410 bases/basepairs, about 360 to about 410 bases/basepairs, about 370 to about 410 bases/basepairs, about 380 to about 410 bases/basepairs, about 390 to about 410 bases/basepairs, about 400 to about 410 bases/basepairs about 350 to about 400 bases/basepairs, about 360 to about 400 bases/basepairs, about 370 to about 400 bases/basepairs, about 380 to about 400 bases/basepairs, about 390 to about 400 bases/basepairs, about 350 to about 390 bases/basepairs, about 360 to about 390 bases/basepairs, about 370 to about 390 bases/basepairs, about 380 to about 390 bases/basepairs, about 350 to about 380 bases/basepairs, about 360 to about 380 bases/basepairs, about 370 to about 380 bases/basepairs, about 350 to about 370 bases/basepairs, about 360 to about 370 bases/basepairs, or about 350 to about 360 bases/basepairs in length.

The methods may further include removing residual RNA from the cDNA prior to amplification. For example, removal of residual RNA can be accomplished by enzymatic methods, hybridization methods, filtration methods, and the like. In some embodiments, the methods further comprise treating the cDNA mixture with an RNase.

In some embodiments, the amplicons are purified prior to sequencing. The purification may comprise size separation, removal of single-stranded nucleic impurities, and the like. In some embodiments, the methods further comprise separating the target amplicons based on size selection following amplicon production. In some embodiments, the methods further comprise removing the single stranded nucleic acids, including unused primers, following amplicon production. In some embodiments, the purification includes enzymatic methods (e.g., exonuclease digestion), hybridization methods, chromatographic methods (e.g., specific affinity columns or beads), filtration methods, and the like.

Once amplified, the amplicons can be subject to any known DNA sequencing technique, including conventional sequencing techniques or next generation sequencing (NGS) techniques. As used herein, the term “next generation sequencing” (NGS) or “high throughput sequencing” refers to the so-called parallel sequencing-by-synthesis or ligation sequencing platform currently employed by Illumina, Life Technologies, Roche, etc. Next generation sequencing methods may also include Nanopore sequencing methods such as commercialized by Oxford Nanopore Technologies, electron detection methods such as Ion Torrent technology commercialized by Life Technologies, and single molecule fluorescence based methods such as commercialized by Pacific Biosciences.

Adaptors can be appended to the end of the amplicons for use during sequencing and the following analysis. In some embodiments, an adaptor comprising a tag (e.g., comprising a barcode sequence) is added to the target amplicon after amplification (e.g., in a ligase reaction, in a subsequent amplification reaction) to produce an identifiable adaptor-amplicon for use in the sequencing reaction. As used herein, an “adaptor” is an oligonucleotide that is linked or is designed to be linked to a nucleic acid to introduce the nucleic acid into a sequencing workflow. An adaptor may be single-stranded or double-stranded (e.g., a double-stranded DNA or a single-stranded DNA). At least a portion of the adaptor comprises a known sequence. Some embodiments of adaptors comprise a marker, index, barcode, tag, or other sequence by which the adaptor and a nucleic acid to which it is linked are identifiable. Exemplary adaptors are shown in FIGS. 1 and 2.

Analysis of the data following NGS techniques can use various commercial programs (e.g., GeneSpring™ from Agilent Technologies) to derive information such as dominant transcript isoforms, relative abundance information, and primary genomic sequence identity by various alignment and quantification methods. In some embodiments, the resulting transcriptomic analysis can in turn be used for proteomic analysis.

In some embodiments, a control is analyzed concurrently with the target, such that results can be compared or validated on the basis of the control. As used herein, the term “control” when used in reference to nucleic acid analysis refers to a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Controls may also be external.

In some embodiments, the method disclosed herein includes use of an external RNA control which is added at any point in the method prior to the amplification. As such, the methods may further comprise adding external RNA control to the sample. When using an external RNA control, the control may be added prior to RNA extraction or prior to reverse transcription and production of cDNA. Accordingly, the amplification may further comprise contacting the sample with a pair of oligonucleotide primers configured to specifically amplify the external RNA control.

In some embodiments, the transcriptomic profiling comprises determining a gene expression or relative gene expression of the target RNAs. Assaying the expression level for a plurality of target genes may comprise the use of an algorithm or classifier. Transcriptomic profiling may further be used to compare transcript sequences to genomic sequences for the subject. Thus, transcriptomic profiling may result in the discovery of alternative transcripts, gene fusions, and allele-specific expression patterns.

In some embodiments, the methods may further comprise quantifying protein levels in the fecal sample corresponding or in addition to those gene targets in the transcriptomic analysis. For example, the methods may comprise determining protein levels for a transcript showing particularly high or low expression.

In some embodiments, the transcriptomic profiling comprises analyzing relationship between gene expression and cellular lineage. For example, gene expression in different tissues or cell types can be determined by conventional methods and compared to the transcriptomic data. Thus, the transcriptomic data can be correlated to certain tissues or cell types.

In some embodiments, the methods comprise correlating the transcriptomic data with gut microbiome data. For example, the methods described herein may be alternatively used to amplify RNA derived from gut bacteria cells found in the fecal sample to determine state of the gut microbiome (e.g., to determine the relative abundance of individual organisms). Alternatively or in addition, the gut microbiome may be profiled by, for example, other microbial transcriptomic approaches, metagenomic approaches (e.g., shotgun sequencing, 16S rRNA-based approaches), culturomic approaches, metabolomic approaches, and combinations thereof. The methods can be combined with monitoring of the gut microbiome over time providing analysis of the correlation and links between microbiota species and gene expression of gastrointestinal tract for increasing the understanding mechanism of host-microbe interactions and developing novel probiotics.

In some embodiments, the methods further comprise obtaining the fecal sample from a subject and processing the sample, as described elsewhere herein, by homogenization, cell lysis, and RNA extraction. In some embodiments, the subject is human. In some embodiments, in the methods disclosed herein, the subject has or is suspected of having a disease or disorder (e.g., gastrointestinal disease or disorder).

The fecal samples may be obtained in a medical facility, e.g., at an Emergency Room, urgent care clinic, walk-in clinic, a long-term care facility, or another appropriate site of medical practice. The subject sample may be obtained in a home or residential setting (e.g., a senior living or hospice setting) and transported to a second site (e.g., laboratory or medical facility) for analysis.

### Monitoring and Diagnosis

Transcriptome profiling using the methods disclosed herein facilitates the analysis of differentially expressed genes as a transcriptional response to different environmental stimuli or physiological/pathological conditions.

Accordingly, the disclosed methods may be used to detect or identify a disease state or disorder of a subject, determine the likelihood that a subject will contract a given disease or disorder, determine the likelihood that a subject with a disease or disorder will respond to therapy, determine the prognosis of a subject with a disease or disorder (or its likely progression or regression), and determine the effect of a treatment on a subject with a disease or disorder. For example, the disclosed methods may be used to determine whether or not a subject is suffering from a given disease or disorder. In some embodiments, the disclosed methods can be used to compare normal healthy subjects with subjects having a disease or disorder. In some embodiments, the disclosed methods can be used to compare subtypes or stages of a disease or disorder.

The disclosed methods, and the resulting transcriptomic data, may also be used in combination with other genomic, epigenomic, proteomic, and/or metabolomic data for the analysis and diagnosis of diseases and disorders, particularly complex diseases and disorders. Thus, the disclosed methods may be used alone or as part of a multi-omic approach to study diseases and disorders, identify biomarkers in diseases and disorders, and aid in the diagnosis of diseases and disorders. For example, the disclosed methods may be used to identify differential expression of a gene or set of genes based on a physiological/pathological condition, which can then be used as biomarkers or for diagnostic methods.

Thus, in some embodiments, in the methods disclosed herein, the subject has or is suspected of having a disease or disorder. The disease or disorder may comprise a gastrointestinal disease or disorder, a metabolic disease or disorder, a neurological disease or disorder, a cardiovascular disease or disorder, an infectious disease or disorder, and/or a respiratory disease or disorder.

In select embodiments, the subject has or is suspected of having a gastrointestinal disease or disorder. Gastrointestinal disease and disorders include a wide range of diseases affecting the esophagus, liver, stomach, small and large intestines, gallbladder, and pancreas. Exemplary gastrointestinal diseases and disorders include, but are not limited to, irritable bowel syndrome (IBS), colitis (e.g., infectious colitis, ulcerative colitis, Crohn's disease, ischemic colitis, radiation colitis), colon polyps and cancer, peptic ulcer disease, gastritis, gastroenteritis, celiac disease, gallstones, fecal incontinence, lactose intolerance, Hirschsprung disease, abdominal adhesions, Barrett's esophagus, appendicitis, indigestion (dyspepsia), intestinal pseudo-obstruction, pancreatitis, short bowel syndrome, Whipple's disease, Zollinger-Ellison syndrome, malabsorption syndromes and hepatitis.

The methods disclosed herein can be used for monitoring progression of a disease or disorder and/or response to treatment. For example, two or more samples are obtained, wherein the two or more samples are separated by a period of time. Specifically, a subsequent sample can be obtained minutes, hours, days, weeks, months, or years after an initial sample was obtained. The transcriptomic profile may be obtained for each of the samples and changes between the fecal samples can be determined. In some embodiments, the changes in the transcriptome profile are associated with progression or regression of the disease or disorder.

In some embodiments, the methods described herein are integrated into a treatment method for a subject. For example, in some embodiments, a subject provides a fecal sample, the fecal sample is analyzed by the methods described herein, a report of the results is generated, and the subject is treated based on the results (e.g., commence a new treatment, continue existing treatment, change in treatment (e.g., change in intervention type, dose, timing, etc.), hospitalization, watchful waiting, etc.).

Treatments for example, may include administering to the subject an effective amount of anti-inflammatory drugs, antibiotics, immune system suppressors, Janus kinase inhibitors, probiotics, biologics (e.g., natalizumab, vedolizumab, infliximab, adalimumab, certolizumab pegol, golimumab, and ustekinumab), analgesics, anti-diarrheals, serotonergic agents, antidepressants, chloride channel activators, chloride channel blockers, guanylate cyclase agonists, opioids, pancreatin, intravenous fluids, an intestinal alkaline phosphatase (iAP) protein replacement composition, parenteral (or intravenous) nutrition (including vitamins and supplements), or a combination thereof.

The disclosed methods may also be used to assess overall gut health or wellness in any subject at a single point in time or monitor gut health or wellness over a longer period of time. There is a known relationship between good gut health and the overall health of a subject and the disclosed methods would provide a way to monitor, promote and take steps to improve gut health in an individual. Overall gut health or wellness can be assessed by evaluating gene functions involved in basic gut physiology, including gut motility, barrier function, bile acid metabolism, and gut-brain signaling. In some embodiments, the subject is a healthy individual. In some embodiments, the subject is not suffering from a gastrointestinal disease or disorder.

In some embodiments, the methods comprise generating a transcriptome profile of subject cells in one or more fecal samples from the subject by the methods disclosed herein and comparing the transcriptome profile to one or more controls to determine a measurement or assessment of overall gut health. In some embodiments, the one or more fecal samples may be separated from each other by a period of time ranging for weeks, months or years.

The assessment can be provided as any type of output (e.g., a score or grade) which is associated with the overall health or condition of the subject's gut. In some embodiments, the methods further comprise preparing the assessment and/or reporting the assessment to the subject. The assessment may further comprise instructions on improving gut health or steps to take to reverse any unwanted changes in gut health. For example, gut management instructions may include diet and nutrition suggestions, food allergy or intolerance information, or information on related health concerns (e.g., weight control, stress management, and the like.)

### Kits or Systems

Also provided herein are systems or kits for carrying out the disclosed methods. In some embodiments, the kit comprises primers or primer pairs specific for a target sequence, for example those described herein in Tables 1 and 2. The primers or pairs of primers are suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100, 110, or more primers or pairs of primers suitable for selectively amplifying one or more targets.

The kit may further comprise reagents for extracting or purifying RNA, amplifying and detecting nucleic acid sequences, and instructions for amplifying and sequencing target sequences. Examples of suitable reagents for inclusion in the kit (in addition to the oligonucleotides described herein) include conventional reagents employed in nucleic acid amplification reactions, such as, for example, one or more enzymes having polymerase activity, enzyme cofactors (such as magnesium or nicotinamide adenine dinucleotide (NAD)), salts, buffers, deoxyribonucleotide, or ribonucleotide triphosphates (dNTPs/rNTPs; for example, deoxyadenosine triphosphate, deoxyguanosine triphosphate, deoxycytidine triphosphate, and deoxythymidine triphosphate) blocking agents, labeling agents, and the like.

The kit may comprise instructions for using the reagents and primers described herein, e.g., for processing the test sample, extracting nucleic acid molecules, and/or performing the test; and for interpreting the results obtained. The instructions may be printed or provided electronically (e.g., DVD, CD, or available for viewing or acquiring via internet resources).

The kit may be supplied in a solid (e.g., lyophilized) or liquid form. The various components of the kit of the present disclosure may optionally be contained within different containers (e.g., vial, ampoule, test tube, flask, or bottle) for each individual component (e.g., amplification oligonucleotides, probe oligonucleotides, or buffer). Each component will generally be suitable as aliquoted in its respective container or provided in a concentrated form. Other containers suitable for conducting certain steps of the amplification/detection assay may also be provided. The individual containers are preferably maintained in close confinement for commercial sale.

### EXAMPLES

The following are examples of the present invention and are not to be construed as limiting.

### Materials and Methods

Fecal RNA extraction Frozen (−80° C.) fecal samples are removed and weighed, if desired. Weights of fecal samples were used to measure absolute abundance values. DNA/RNA Shield buffer (500-1000 μL) was added to the sample while the fecal sample was still frozen. The fecal samples were incubated on ice to thaw for at least 30 minutes. Glass beads were added to each sample or sample aliquot and mechanical beating with the beads was used to homogenize the fecal sample. Following homogenization, the sample is left on ice for 30 minutes, to lyse fragile host cells and release RNA into solution, while minimizing lysis of any microbial cells in the sample. To remove cellular and dietary debris, the incubated sample was centrifuged at 4300 g for 5 minutes at 4° C., and the supernatant was retained. Optionally, external RNA control (ERCC) was added into each sample supernatant. For each pellet of mice feces input, 10 μL of 0.01 ng/μL ERCC was added.

RNA extraction was completed using standard Direct-zol RNA extraction according to manufacturer's protocols. TRIzol reagent (600 μL) was added to each sample supernatant, mixed by inversion, and incubated at room temperature for 15 to 30 minutes for cell lysis. Following removal of solid contaminants by centrifugation for 10 minutes at 4° C., an equal part of 100% ethanol was added to the resulting sample and mixed. RNA was purified using Zymo Direct-zol RNA Miniprep Kit with DNase I treatment. The concentration of RNA is measured by Quant-iT BR RNA kit with 2 μL as input. Extracted RNA can be stored at −80° C. if necessary. All downstream steps were completed using a normalized RNA concentration (600 to 1200 ng) to generate a similar amount of library per sample.

Genomic DNA Removal Genomic DNA removal was completed by Turbo DNase. Approximately 500 ng of extracted RNA was mixed with buffer and 2 μL Turbo DNase and incubated for 20 minutes at 37° C. RNase-free solid-phase reversible immobilization (SPRI) beads, were used to purify the resulting RNA.

cDNA generation cDNA was generated from the extracted fecal RNA using a high-yield reverse transcriptase with random hexamer primers. All the resulting RNA following genomic DNA removal was added to a master reaction mix comprising 50 μM random hexamer and 10 mM of each dNTP. The reaction mix was heated at 65° C. for 5 minutes and immediately put on ice for at least 1 minute. After incubation on ice, the reverse transcriptase mix (4 μL 5×SSIV buffer, 1 uL DTT 100 mM, 1 μL RNase inhibitor, 1 uL SSIV enzyme) was added and incubated at 23° C. for 10 minutes, 55° C. for 20 minutes, and 80° C. for 10 minutes. RNase H was added and the resulting mixture was incubated at 37° C. for 20 minutes. cDNA was purified and separated with 2.4×SPRI beads cleanup and elution in nuclease-free water.

Amplicon-generation Multiplex PCR was used to amplify targeted regions of genes. Multiplex PCR was carried out using 5 μL cDNA, 0.1 μM of each primer, including primer to external control RNA if used, 2 μL Taq polymerase-based Multiplex PCR 5× Master Mix, DMSO in water. The PCR methodology was as follows: 95° C. for 2 min; cycles of 95° C. for 30 sec, 61° C. for 30 sec, and 68° C. for 1 min; and 68° C. for 5 min. A limited number of cycles was used, stopping the reaction at exponential phase. For mouse samples, 14 cycles were used, whereas for human samples, 20 cycles were used. PCR product can be stored frozen until purification can be completed. The resulting amplicons were purified with enzymatic digestion (exonuclease digestion) and rigorous SPRI bead-based size selection. PCR product or purified amplicons can be stored frozen.

Adaptor addition A second PCR amplification was applied to the purified amplicons to add Indexed Illumina sequencing adapter for sequencing. The PCR reaction includes high-yield KAPA PCR master mix with 10 μM barcoded P5 and P7 primers. The PCR methodology was as follows: 98° C. for 3 min; 30 cycles of 98° C. for 20 sec, 67° C. for 15 sec, and 72° C. for 1 min; and 72° C. for 5 min. The resulting amplicons were purified with SPRI beads and gel electrophoresis-based size selection to create libraries of target amplicons.

Library Sequencing Sequencing reactions were carried out on an Illumina NextSeq system following manufacturer's instructions with an aim of 1 million reads per sample: mid-output mode: 150M reads, 150 cycle, 76 bp×2, pair-end, 15 hr; high-output mode: 400M reads, 150 cycle, 76 bp×2, pair-end, 18 hr.

TABLE 1
Mouse Gene Targets and Primers

| | Forward | Reverse | Total |
|---|---|---|---|
| | Primer | Primer | amplicon |
| Gene | SEQ ID NO | SEQ ID NO | size (bp) |
| Gnai3 | 1 | 535 | 417 |
| Narf | 2 | 536 | 392 |
| Ngfr | 3 | 537 | 406 |
| Xpo6 | 4 | 538 | 450 |
| Tfe3 | 5 | 539 | 406 |
| Brat1 | 6 | 540 | 443 |
| Gna12 | 7 | 541 | 449 |
| Dlat | 8 | 542 | 391 |
| Sdhd | 9 | 543 | 436 |
| Gpr107 | 10 | 544 | 429 |
| Nalcn | 11 | 545 | 426 |
| Btbd17 | 12 | 546 | 442 |
| Slfn4 | 13 | 547 | 416 |
| Lhx2 | 14 | 548 | 448 |
| Clec10a | 15 | 549 | 393 |
| Rtca | 16 | 550 | 438 |
| Dbt | 17 | 551 | 396 |
| Dazap2 | 18 | 552 | 434 |
| Trappc10 | 19 | 553 | 439 |
| Tbrg4 | 20 | 554 | 421 |
| Ndufa9 | 21 | 555 | 450 |
| Pparg | 22 | 556 | 417 |
| Pdgfb | 23 | 557 | 428 |
| Acvrl1 | 24 | 558 | 448 |
| Acvr1b | 25 | 559 | 448 |
| Gabra2 | 26 | 560 | 423 |
| Sez6 | 27 | 561 | 424 |
| Cd52 | 28 | 562 | 412 |
| Il12rb1 | 29 | 563 | 445 |
| Car4 | 30 | 564 | 441 |
| Il4 | 31 | 565 | 434 |
| Mmp14 | 32 | 566 | 406 |
| Ccl3 | 33 | 567 | 440 |
| Timp1 | 34 | 568 | 448 |
| Slc26a3 | 35 | 569 | 411 |
| Sema6b | 36 | 570 | 449 |
| Gabrg1 | 37 | 571 | 409 |
| Ube2c | 38 | 572 | 401 |
| Tbx21 | 39 | 573 | 417 |
| Cldn15 | 40 | 574 | 396 |
| Il16 | 41 | 575 | 416 |
| Vwf | 42 | 576 | 419 |
| Pdzd4 | 43 | 577 | 401 |
| Ncan | 44 | 578 | 413 |
| Rgs20 | 45 | 579 | 416 |
| Tgfb1 | 46 | 580 | 420 |
| Il17ra | 47 | 581 | 396 |
| Ebi3 | 48 | 582 | 426 |
| Edar | 49 | 583 | 397 |
| Hck | 50 | 584 | 420 |
| Cd79a | 51 | 585 | 392 |
| Has1 | 52 | 586 | 445 |
| Il7r | 53 | 587 | 409 |
| Stat3 | 54 | 588 | 432 |
| Stat5a | 55 | 589 | 414 |
| Psap | 56 | 590 | 447 |
| Il12b | 57 | 591 | 417 |
| Il11 | 58 | 592 | 446 |
| Ccl24 | 59 | 593 | 394 |
| Cd44 | 60 | 594 | 417 |
| Prlr | 61 | 595 | 405 |
| Il5ra | 62 | 596 | 413 |
| Kit | 63 | 597 | 430 |
| Tnfsf14 | 64 | 598 | 399 |
| Epor | 65 | 599 | 442 |
| Mpl | 66 | 600 | 437 |
| Adamts4 | 67 | 601 | 419 |
| Sod2 | 68 | 602 | 391 |
| Tgfbr1 | 69 | 603 | 403 |
| Dio2 | 70 | 604 | 400 |
| Relt | 71 | 605 | 435 |
| Bmp7 | 72 | 606 | 417 |
| Ccl8 | 73 | 607 | 414 |
| Mpo | 74 | 608 | 417 |
| Met | 75 | 609 | 424 |
| Tnfrsf13b | 76 | 610 | 426 |
| Slc5a1 | 77 | 611 | 421 |
| Lypd8 | 78 | 612 | 426 |
| 1810065E05Rik | 79 | 613 | 408 |
| Gip | 80 | 614 | 430 |
| Csf1 | 81 | 615 | 449 |
| Ace2 | 82 | 616 | 420 |
| Gzmb | 83 | 617 | 415 |
| Gata3 | 84 | 618 | 394 |
| Prpsap1 | 85 | 619 | 419 |
| Fcgr1 | 86 | 620 | 407 |
| Il17rb | 87 | 621 | 430 |
| Lbp | 88 | 622 | 443 |
| Il19 | 89 | 623 | 417 |
| Il10 | 90 | 624 | 419 |
| Slpi | 91 | 625 | 448 |
| Il13ra1 | 92 | 626 | 410 |
| Pyy | 93 | 627 | 440 |
| Ppy | 94 | 628 | 437 |
| Cd40 | 95 | 629 | 412 |
| Hnf4g | 96 | 630 | 403 |
| Ada | 97 | 631 | 437 |
| Mmp9 | 98 | 632 | 441 |
| Il12rb2 | 99 | 633 | 450 |
| Mmp7 | 100 | 634 | 412 |
| Il3 | 101 | 635 | 424 |
| Csf2 | 102 | 636 | 438 |
| Tm4sf5 | 103 | 637 | 424 |
| Cxcl16 | 104 | 638 | 428 |
| Cc14 | 105 | 639 | 403 |
| Cd70 | 106 | 640 | 435 |
| Mydgf | 107 | 641 | 450 |
| Nts | 108 | 642 | 449 |
| Enpp3 | 109 | 643 | 415 |
| Il20ra | 110 | 644 | 415 |
| Ifngr1 | 111 | 645 | 406 |
| Hkdc1 | 112 | 646 | 403 |
| Egfr | 113 | 647 | 390 |
| Lgr5 | 114 | 648 | 395 |
| 9530003J23Rik | 115 | 649 | 446 |
| Slc5a4b | 116 | 650 | 417 |
| Slc5a4a | 117 | 651 | 395 |
| Il9r | 118 | 652 | 390 |
| Madcam1 | 119 | 653 | 446 |
| Flt4 | 120 | 654 | 416 |
| Il13 | 121 | 655 | 437 |
| Upp1 | 122 | 656 | 412 |
| Npc1l1 | 123 | 657 | 446 |
| Slc7a15 | 124 | 658 | 402 |
| Tpo | 125 | 659 | 390 |
| Ccl11 | 126 | 660 | 400 |
| Ccl1 | 127 | 661 | 432 |
| Slc9a3r1 | 128 | 662 | 397 |
| Nos2 | 129 | 663 | 406 |
| Tmigd1 | 130 | 664 | 419 |
| Krt19 | 131 | 665 | 448 |
| Serpina3n | 132 | 666 | 448 |
| Arg2 | 133 | 667 | 413 |
| Ifi27l2b | 134 | 668 | 416 |
| Tubal3 | 135 | 669 | 419 |
| Tgfb3 | 136 | 670 | 394 |
| Exoc3l4 | 137 | 671 | 411 |
| Prl | 138 | 672 | 400 |
| Cxcl14 | 139 | 673 | 423 |
| Il9 | 140 | 674 | 414 |
| Ocln | 141 | 675 | 447 |
| Il6st | 142 | 676 | 425 |
| Bmpr1a | 143 | 677 | 434 |
| Tnfsf11 | 144 | 678 | 435 |
| Olfm4 | 145 | 679 | 446 |
| Tnfrsf10b | 146 | 680 | 444 |
| Acod1 | 147 | 681 | 422 |
| Osmr | 148 | 682 | 420 |
| Tnfrsf17 | 149 | 683 | 450 |
| Cldn1 | 150 | 684 | 441 |
| Il1rap | 151 | 685 | 409 |
| Apod | 152 | 686 | 428 |
| Ly6g | 153 | 687 | 405 |
| Retnlb | 154 | 688 | 416 |
| Retnlg | 155 | 689 | 409 |
| Cd86 | 156 | 690 | 427 |
| Ifngr2 | 157 | 691 | 401 |
| Ifnar1 | 158 | 692 | 437 |
| Il10rb | 159 | 693 | 426 |
| Ifnar2 | 160 | 694 | 395 |
| Cela1 | 161 | 695 | 431 |
| Krt18 | 162 | 696 | 410 |
| Amhr2 | 163 | 697 | 427 |
| Fabp2 | 164 | 698 | 403 |
| Cxcl13 | 165 | 699 | 407 |
| Gzma | 166 | 700 | 419 |
| Reg2 | 167 | 701 | 446 |
| Cpn2 | 168 | 702 | 426 |
| Il15ra | 169 | 703 | 398 |
| Ccl25 | 170 | 704 | 435 |
| Cd4 | 171 | 705 | 436 |
| Tnfrsf12a | 172 | 706 | 419 |
| Tnfrsf21 | 173 | 707 | 405 |
| Vegfa | 174 | 708 | 420 |
| Tff3 | 175 | 709 | 411 |
| Tff1 | 176 | 710 | 401 |
| C3 | 177 | 711 | 422 |
| Tslp | 178 | 712 | 422 |
| Ltb | 179 | 713 | 442 |
| Tnf | 180 | 714 | 398 |
| Lta | 181 | 715 | 410 |
| Il17b | 182 | 716 | 392 |
| Pdgfrb | 183 | 717 | 396 |
| Csf1r | 184 | 718 | 422 |
| Ms4a4c | 185 | 719 | 392 |
| Ms4a6b | 186 | 720 | 405 |
| Ms4a6d | 187 | 721 | 412 |
| Slc15a3 | 188 | 722 | 391 |
| Fas | 189 | 723 | 448 |
| Jak2 | 190 | 724 | 402 |
| Tnfrsf25 | 191 | 725 | 448 |
| Il33 | 192 | 726 | 418 |
| Vegfb | 193 | 727 | 446 |
| Cyp2c55 | 194 | 728 | 404 |
| Cd7 | 195 | 729 | 443 |
| Cpn1 | 196 | 730 | 430 |
| Il23a | 197 | 731 | 408 |
| Inhbc | 198 | 732 | 409 |
| St8sia5 | 199 | 733 | 433 |
| Muc2 | 200 | 734 | 440 |
| Il6 | 201 | 735 | 448 |
| Ccr1 | 202 | 736 | 422 |
| Pdgfa | 203 | 737 | 424 |
| Casp1 | 204 | 738 | 445 |
| Il17a | 205 | 739 | 423 |
| Ccdc150 | 206 | 740 | 399 |
| Cps1 | 207 | 741 | 404 |
| Ikzf2 | 208 | 742 | 397 |
| Ctla4 | 209 | 743 | 443 |
| Il18rap | 210 | 744 | 407 |
| Ilrl1 | 211 | 745 | 422 |
| Il18r1 | 212 | 746 | 397 |
| Il1r1 | 213 | 747 | 419 |
| Il1r2 | 214 | 748 | 418 |
| Stat1 | 215 | 749 | 409 |
| Ccl20 | 216 | 750 | 398 |
| Cxcr2 | 217 | 751 | 396 |
| Ptma | 218 | 752 | 394 |
| Atg16l1 | 219 | 753 | 421 |
| Thfrsf11a | 220 | 754 | 427 |
| Marco | 221 | 755 | 428 |
| Cd55 | 222 | 756 | 424 |
| Cd55b | 223 | 757 | 438 |
| Il20 | 224 | 758 | 425 |
| Il24 | 225 | 759 | 439 |
| Mptx1 | 226 | 760 | 420 |
| Ifi211 | 227 | 761 | 448 |
| Xcl1 | 228 | 762 | 394 |
| Selp | 229 | 763 | 430 |
| Sele | 230 | 764 | 437 |
| Cr2 | 231 | 765 | 407 |
| Fcgr2b | 232 | 766 | 405 |
| Tnfsf4 | 233 | 767 | 408 |
| Vim | 234 | 768 | 398 |
| Bmi1 | 235 | 769 | 395 |
| Il2ra | 236 | 770 | 398 |
| St6galnac6 | 237 | 771 | 391 |
| Lcn2 | 238 | 772 | 422 |
| Acvr1 | 239 | 773 | 396 |
| Hc | 240 | 774 | 411 |
| Il1rn | 241 | 775 | 436 |
| Chgb | 242 | 776 | 422 |
| Bmp2 | 243 | 777 | 423 |
| Hdc | 244 | 778 | 420 |
| Il1b | 245 | 779 | 415 |
| Il1a | 246 | 780 | 398 |
| Cpxm1 | 247 | 781 | 403 |
| Zbp1 | 248 | 782 | 411 |
| Car1 | 249 | 783 | 402 |
| Il21 | 250 | 784 | 407 |
| Il2 | 251 | 785 | 434 |
| Il12a | 252 | 786 | 449 |
| Ptx3 | 253 | 787 | 399 |
| Ptpn22 | 254 | 788 | 429 |
| Reg4 | 255 | 789 | 443 |
| Vcam1 | 256 | 790 | 449 |
| Tlr2 | 257 | 791 | 412 |
| Egf | 258 | 792 | 415 |
| Pdgfc | 259 | 793 | 416 |
| Rorc | 260 | 794 | 429 |
| Clca1 | 261 | 795 | 393 |
| Gbp2 | 262 | 796 | 448 |
| Cga | 263 | 797 | 402 |
| Tnfsf8 | 264 | 798 | 442 |
| Cntfr | 265 | 799 | 434 |
| Tnfrsf1b | 266 | 800 | 442 |
| Tnfrsf8 | 267 | 801 | 449 |
| Tspan1 | 268 | 802 | 438 |
| Alpl | 269 | 803 | 447 |
| Stmn1 | 270 | 804 | 410 |
| Csf3r | 271 | 805 | 407 |
| Hgf | 272 | 806 | 408 |
| Tnfrsf9 | 273 | 807 | 399 |
| Abcb1b | 274 | 808 | 413 |
| Tnfrsf4 | 275 | 809 | 394 |
| Plb1 | 276 | 810 | 411 |
| Pdgfra | 277 | 811 | 397 |
| Slc10a6 | 278 | 812 | 445 |
| Cxcl5 | 279 | 813 | 414 |
| Ppbp | 280 | 814 | 431 |
| Pf4 | 281 | 815 | 443 |
| Ereg | 282 | 816 | 398 |
| Cxcl3 | 283 | 817 | 416 |
| Cxcl1 | 284 | 818 | 438 |
| Cxcl9 | 285 | 819 | 428 |
| Il31 | 286 | 820 | 406 |
| Ccr9 | 287 | 821 | 397 |
| Rasl11a | 288 | 822 | 411 |
| Cdx2 | 289 | 823 | 442 |
| Flt1 | 290 | 824 | 392 |
| Col1a2 | 291 | 825 | 399 |
| Epo | 292 | 826 | 417 |
| Gpnmb | 293 | 827 | 391 |
| Sval1 | 294 | 828 | 420 |
| Reg3g | 295 | 829 | 418 |
| Clec4e | 296 | 830 | 396 |
| Clec4d | 297 | 831 | 427 |
| Klrk1 | 298 | 832 | 446 |
| Cd27 | 299 | 833 | 428 |
| Ltbr | 300 | 834 | 426 |
| Tnfrsf1a | 301 | 835 | 403 |
| Tip1 | 302 | 836 | 433 |
| Tyrobp | 303 | 837 | 428 |
| Gm45837 | 304 | 838 | 410 |
| Coro1a | 305 | 839 | 396 |
| Chrdl2 | 306 | 840 | 439 |
| Il21r | 307 | 841 | 390 |
| Aqp8 | 308 | 842 | 414 |
| Pvcard | 309 | 843 | 440 |
| Oat | 310 | 844 | 450 |
| Slc6a14 | 311 | 845 | 409 |
| Cd40lg | 312 | 846 | 395 |
| Nox1 | 313 | 847 | 421 |
| Il2rg | 314 | 848 | 439 |
| Vegfd | 315 | 849 | 399 |
| Irak1 | 316 | 850 | 403 |
| F10 | 317 | 851 | 416 |
| Lamp1 | 318 | 852 | 402 |
| Tnfsf13b | 319 | 853 | 426 |
| Vegfc | 320 | 854 | 434 |
| Ido1 | 321 | 855 | 437 |
| Il15 | 322 | 856 | 394 |
| Hp | 323 | 857 | 397 |
| Il34 | 324 | 858 | 441 |
| Mt1 | 325 | 859 | 449 |
| Cx3cl1 | 326 | 860 | 390 |
| Cc122 | 327 | 861 | 425 |
| Ccl17 | 328 | 862 | 403 |
| Tirap | 329 | 863 | 393 |
| Il10ra | 330 | 864 | 432 |
| Tyk2 | 331 | 865 | 443 |
| Tgfbr2 | 332 | 866 | 441 |
| Ptgs2 | 333 | 867 | 434 |
| Ltf | 334 | 868 | 400 |
| Myd88 | 335 | 869 | 405 |
| Crlf2 | 336 | 870 | 390 |
| Egr3 | 337 | 871 | 415 |
| Slc26a2 | 338 | 872 | 416 |
| Lif | 339 | 873 | 414 |
| Eda2r | 340 | 874 | 446 |
| Ly6d | 341 | 875 | 397 |
| Cxcl10 | 342 | 876 | 448 |
| Fam151a | 343 | 877 | 439 |
| Ccl5 | 344 | 878 | 424 |
| Ubd | 345 | 879 | 390 |
| Amh | 346 | 880 | 412 |
| Ccl12 | 347 | 881 | 394 |
| Ccl7 | 348 | 882 | 430 |
| Ccl2 | 349 | 883 | 415 |
| Cer3 | 350 | 884 | 400 |
| Tnfsf9 | 351 | 885 | 429 |
| Isg15 | 352 | 886 | 426 |
| Il5 | 353 | 887 | 436 |
| Leap2 | 354 | 888 | 429 |
| Clca3b | 355 | 889 | 409 |
| Inhbb | 356 | 890 | 440 |
| Lrg1 | 357 | 891 | 392 |
| Lypd8l | 358 | 892 | 400 |
| Il22ra1 | 359 | 893 | 443 |
| Icam1 | 360 | 894 | 441 |
| Serpine1 | 361 | 895 | 420 |
| Cer7 | 362 | 896 | 390 |
| Csf3 | 363 | 897 | 421 |
| Itln1 | 364 | 898 | 400 |
| Gdf5 | 365 | 899 | 409 |
| Cd160 | 366 | 900 | 394 |
| Sct | 367 | 901 | 430 |
| Tlr4 | 368 | 902 | 430 |
| Il18 | 369 | 903 | 402 |
| Tgfb2 | 370 | 904 | 416 |
| Lgi2 | 371 | 905 | 450 |
| Tnfsf10 | 372 | 906 | 391 |
| Il22ra2 | 373 | 907 | 438 |
| Ptger4 | 374 | 908 | 433 |
| Saa3 | 375 | 909 | 394 |
| I17 | 376 | 910 | 395 |
| Abcb1a | 377 | 911 | 396 |
| Clcf1 | 378 | 912 | 392 |
| 1l25 | 379 | 913 | 408 |
| Chil3 | 380 | 914 | 432 |
| Ccr6 | 381 | 915 | 439 |
| Inhba | 382 | 916 | 409 |
| Bdkrb1 | 383 | 917 | 406 |
| Serpina3g | 384 | 918 | 390 |
| 1l17f | 385 | 919 | 401 |
| Tnfrsf18 | 386 | 920 | 391 |
| Ccr8 | 387 | 921 | 413 |
| Tnfrsf14 | 388 | 922 | 421 |
| Ctf1 | 389 | 923 | 414 |
| Muc1 | 390 | 924 | 427 |
| Flt3 | 391 | 925 | 450 |
| Wfdc12 | 392 | 926 | 400 |
| Ifnk | 393 | 927 | 426 |
| Mmp3 | 394 | 928 | 400 |
| Ccr10 | 395 | 929 | 431 |
| Il20rb | 396 | 930 | 420 |
| Il27 | 397 | 931 | 390 |
| Defb1 | 398 | 932 | 406 |
| Tlr9 | 399 | 933 | 390 |
| Ifne | 400 | 934 | 407 |
| Cxcr4 | 401 | 935 | 399 |
| Calhm6 | 402 | 936 | 398 |
| Il17c | 403 | 937 | 432 |
| Bst2 | 404 | 938 | 413 |
| Cd24a | 405 | 939 | 395 |
| H2bc6 | 406 | 940 | 443 |
| Ptgs1 | 407 | 941 | 440 |
| Inhbe | 408 | 942 | 412 |
| Mmp10 | 409 | 943 | 420 |
| Fcgbp | 410 | 944 | 390 |
| Cxcr5 | 411 | 945 | 390 |
| Ccr4 | 412 | 946 | 396 |
| Col6a3 | 413 | 947 | 405 |
| Cxcr1 | 414 | 948 | 397 |
| Cxcr6 | 415 | 949 | 421 |
| Ifnb1 | 416 | 950 | 412 |
| Il23r | 417 | 951 | 449 |
| Ccr2 | 418 | 952 | 435 |
| Zg16 | 419 | 953 | 391 |
| Il17d | 420 | 954 | 436 |
| Cxcr3 | 421 | 955 | 395 |
| Tnfsf15 | 422 | 956 | 391 |
| Mmp13 | 423 | 957 | 446 |
| Plekho2 | 424 | 958 | 411 |
| Siglech | 425 | 959 | 420 |
| Acvr2a | 426 | 960 | 407 |
| Cx3cr1 | 427 | 961 | 419 |
| Cemip | 428 | 962 | 438 |
| Bmpr1b | 429 | 963 | 408 |
| Sh2d6 | 430 | 964 | 403 |
| Socs3 | 431 | 965 | 448 |
| Iigp1 | 432 | 966 | 424 |
| Lifr | 433 | 967 | 433 |
| Fabp1 | 434 | 968 | 416 |
| Hmgb2 | 435 | 969 | 436 |
| Ifng | 436 | 970 | 409 |
| Ghr | 437 | 971 | 392 |
| Nod2 | 438 | 972 | 421 |
| Cyp3a11 | 439 | 973 | 406 |
| S100a8 | 440 | 974 | 416 |
| S100a9 | 441 | 975 | 399 |
| Nfil3 | 442 | 976 | 446 |
| Gapdh | 443 | 977 | 434 |
| Lepr | 444 | 978 | 430 |
| Cxcl2 | 445 | 979 | 422 |
| Fcer1g | 446 | 980 | 397 |
| Osm | 447 | 981 | 431 |
| Fcgr4 | 448 | 982 | 443 |
| Ifitm6 | 449 | 983 | 449 |
| Ifnl2 | 450 | 984 | 440 |
| Lep | 451 | 985 | 409 |
| Defb4 | 452 | 986 | 401 |
| Csf2ra | 453 | 987 | 419 |
| Eda | 454 | 988 | 420 |
| Reg1 | 455 | 989 | 419 |
| Cxcl11 | 456 | 990 | 395 |
| Cxcl17 | 457 | 991 | 400 |
| Xcr1 | 458 | 992 | 390 |
| Tnfrsf19 | 459 | 993 | 434 |
| Ifnl3 | 460 | 994 | 409 |
| Cxcl12 | 461 | 995 | 444 |
| Acvr2b | 462 | 996 | 410 |
| Ifnlr1 | 463 | 997 | 395 |
| Gm49339 | 464 | 998 | 418 |
| Kdr | 465 | 999 | 406 |
| Ifna13 | 466 | 1000 | 414 |
| Ly6m | 467 | 1001 | 399 |
| Tnfrsf11b | 468 | 1002 | 417 |
| Defa24 | 469 | 1003 | 395 |
| Serpina3f | 470 | 1004 | 401 |
| Tnfsf18 | 471 | 1005 | 448 |
| Jchain | 472 | 1006 | 448 |
| Bmpr2 | 473 | 1007 | 410 |
| Cyp2d9 | 474 | 1008 | 439 |
| Tnfrsf13c | 475 | 1009 | 414 |
| Il2rb | 476 | 1010 | 444 |
| Clca4a | 477 | 1011 | 415 |
| Il3ra | 478 | 1012 | 427 |
| Lyz1 | 479 | 1013 | 423 |
| Lyz2 | 480 | 1014 | 422 |
| Wfdc17 | 481 | 1015 | 437 |
| Il18bp | 482 | 1016 | 431 |
| Ifna4 | 483 | 1017 | 446 |
| Ccl19 | 484 | 1018 | 393 |
| Reg3b | 485 | 1019 | 390 |
| Csf2rb | 486 | 1020 | 399 |
| Gm14698 | 487 | 1021 | 402 |
| C4b | 488 | 1022 | 400 |
| Pappa2 | 489 | 1023 | 447 |
| Gm13304 | 490 | 1024 | 410 |
| Gsta1 | 491 | 1025 | 391 |
| Clca4b | 492 | 1026 | 415 |
| Defa22 | 493 | 1027 | 394 |
| Defa30 | 494 | 1028 | 390 |
| Defa21 | 495 | 1029 | 394 |
| Sirpb1c | 496 | 1030 | 442 |
| Il22 | 497 | 1031 | 410 |
| Ccl28 | 498 | 1032 | 402 |
| Grem1 | 499 | 1033 | 397 |
| Cd80 | 500 | 1034 | 415 |
| Ly6a | 501 | 1035 | 404 |
| Trbc2 | 502 | 1036 | 442 |
| Igkc | 503 | 1037 | 394 |
| Ifna2 | 504 | 1038 | 400 |
| Ifna16 | 505 | 1039 | 428 |
| Gm9733 | 506 | 1040 | 437 |
| Gm14288 | 507 | 1041 | 403 |
| Tgtp2 | 508 | 1042 | 399 |
| Tgtp1 | 509 | 1043 | 399 |
| Serpina3m | 510 | 1044 | 420 |
| Ccr5 | 511 | 1045 | 415 |
| Gm43302 | 512 | 1046 | 407 |
| Cntf | 513 | 1047 | 442 |
| Tnfsf13 | 514 | 1048 | 408 |
| Ugt1a8 | 515 | 1049 | 432 |
| Bcl2a1b | 516 | 1050 | 443 |
| Pakap | 517 | 1051 | 414 |
| Ugt1a9 | 518 | 1052 | 432 |
| Iltifb | 519 | 1053 | 398 |
| Gm20458 | 520 | 1054 | 436 |
| Igha | 521 | 1055 | 421 |
| Ifna1 | 522 | 1056 | 391 |
| Sirpb1a | 523 | 1057 | 449 |
| Ifna14 | 524 | 1058 | 446 |
| Gm5849 | 525 | 1059 | 444 |
| Ifna5 | 526 | 1060 | 392 |
| Tnfsf12 | 527 | 1061 | 411 |
| Ifna7 | 528 | 1062 | 432 |
| Ifna6 | 529 | 1063 | 392 |
| Ms4a4a | 530 | 1064 | 393 |
| Gbp6 | 531 | 1065 | 410 |
| Gbp5 | 532 | 1066 | 417 |
| Lilr4b | 533 | 1067 | 427 |
| Lilrb4a | 534 | 1068 | 418 |

TABLE 2
Human Gene Targets and Primers

| | Forward | Reverse | Total |
|---|---|---|---|
| | Primer | Primer | amplicon |
| Gene | SEQ ID NO | SEQ ID NO | size (bp) |
| NKX2-3 | 1069 | 1612 | 449 |
| MYD88 | 1070 | 1613 | 409 |
| GP2 | 1071 | 1614 | 391 |
| ACVR2A | 1072 | 1615 | 417 |
| IL22RA1 | 1073 | 1616 | 419 |
| HPRT1 | 1074 | 1617 | 390 |
| IL18RAP | 1075 | 1618 | 449 |
| RAC2 | 1076 | 1619 | 437 |
| TGFBR1 | 1077 | 1620 | 417 |
| IL7R | 1078 | 1621 | 430 |
| IL37 | 1079 | 1622 | 396 |
| BAX | 1080 | 1623 | 409 |
| TP53 | 1081 | 1624 | 426 |
| IFNGR1 | 1082 | 1625 | 404 |
| TLR1 | 1083 | 1626 | 403 |
| LTA | 1084 | 1627 | 426 |
| CD27 | 1085 | 1628 | 410 |
| NEUROD1 | 1086 | 1629 | 413 |
| SLC4A4 | 1087 | 1630 | 438 |
| CCR1 | 1088 | 1631 | 400 |
| IL3 | 1089 | 1632 | 394 |
| CCR7 | 1090 | 1633 | 436 |
| IL6ST | 1091 | 1634 | 440 |
| PUS10 | 1092 | 1635 | 437 |
| CNTF | 1093 | 1636 | 399 |
| NPW | 1094 | 1637 | 400 |
| CCL20 | 1095 | 1638 | 405 |
| BATF | 1096 | 1639 | 409 |
| HMOX1 | 1097 | 1640 | 444 |
| IL1RL1 | 1098 | 1641 | 422 |
| CFLAR | 1099 | 1642 | 424 |
| CSF2 | 1100 | 1643 | 434 |
| ICAM3 | 1101 | 1644 | 428 |
| VIM | 1102 | 1645 | 433 |
| ZG16 | 1103 | 1646 | 438 |
| DEFA5 | 1104 | 1647 | 430 |
| NLRX1 | 1105 | 1648 | 429 |
| BMP7 | 1106 | 1649 | 391 |
| ABCB1 | 1107 | 1650 | 413 |
| NOD1 | 1108 | 1651 | 404 |
| NLRP12 | 1109 | 1652 | 409 |
| PCNA | 1110 | 1653 | 438 |
| CCL5 | 1111 | 1654 | 412 |
| LTB | 1112 | 1655 | 437 |
| IL10RA | 1113 | 1656 | 428 |
| IL31RA | 1114 | 1657 | 402 |
| TGFB1 | 1115 | 1658 | 411 |
| IL13 | 1116 | 1659 | 398 |
| IL11RA | 1117 | 1660 | 447 |
| TNFRSF19 | 1118 | 1661 | 412 |
| SLC22A5 | 1119 | 1662 | 440 |
| CD70 | 1120 | 1663 | 405 |
| IL27 | 1121 | 1664 | 449 |
| ITGB1 | 1122 | 1665 | 427 |
| ACTB | 1123 | 1666 | 412 |
| EDA2R | 1124 | 1667 | 429 |
| AKT1 | 1125 | 1668 | 425 |
| LACC1 | 1126 | 1669 | 401 |
| CD80 | 1127 | 1670 | 445 |
| PRDM1 | 1128 | 1671 | 428 |
| CRP | 1129 | 1672 | 416 |
| SELENBP1 | 1130 | 1673 | 432 |
| WFDC3 | 1131 | 1674 | 402 |
| AIM2 | 1132 | 1675 | 439 |
| IL9 | 1133 | 1676 | 422 |
| ATG4A | 1134 | 1677 | 392 |
| SLC16A1 | 1135 | 1678 | 431 |
| BMPR1A | 1136 | 1679 | 403 |
| IFNW1 | 1137 | 1680 | 396 |
| FABP2 | 1138 | 1681 | 399 |
| IFNG | 1139 | 1682 | 436 |
| RIPK2 | 1140 | 1683 | 398 |
| IFNAR2 | 1141 | 1684 | 414 |
| FRZB | 1142 | 1685 | 398 |
| CCL13 | 1143 | 1686 | 395 |
| BCL2 | 1144 | 1687 | 401 |
| RUNX3 | 1145 | 1688 | 433 |
| LIF | 1146 | 1689 | 401 |
| PIK3CA | 1147 | 1690 | 423 |
| IL11 | 1148 | 1691 | 402 |
| ITGAX | 1149 | 1692 | 396 |
| LRP6 | 1150 | 1693 | 426 |
| MEFV | 1151 | 1694 | 412 |
| ECM1 | 1152 | 1695 | 445 |
| TIGIT | 1153 | 1696 | 397 |
| BAHD1 | 1154 | 1697 | 398 |
| CCL14 | 1155 | 1698 | 449 |
| CCL19 | 1156 | 1699 | 449 |
| POU2F3 | 1157 | 1700 | 390 |
| ORMDL3 | 1158 | 1701 | 443 |
| LGR5 | 1159 | 1702 | 426 |
| FABP5 | 1160 | 1703 | 438 |
| TNFRSF13C | 1161 | 1704 | 403 |
| ANK3 | 1162 | 1705 | 440 |
| XBP1 | 1163 | 1706 | 418 |
| TLR5 | 1164 | 1707 | 397 |
| MUC2 | 1165 | 1708 | 410 |
| SPIB | 1166 | 1709 | 423 |
| ACVR1B | 1167 | 1710 | 395 |
| LCK | 1168 | 1711 | 409 |
| ITGAM | 1169 | 1712 | 403 |
| IL17RA | 1170 | 1713 | 399 |
| VWF | 1171 | 1714 | 395 |
| ATG16L2 | 1172 | 1715 | 414 |
| FADS2 | 1173 | 1716 | 432 |
| GSDMB | 1174 | 1717 | 409 |
| CD19 | 1175 | 1718 | 450 |
| CLCF1 | 1176 | 1719 | 431 |
| CXCL12 | 1177 | 1720 | 405 |
| TNFRSF10A | 1178 | 1721 | 418 |
| REL | 1179 | 1722 | 403 |
| IL17C | 1180 | 1723 | 438 |
| RBP2 | 1181 | 1724 | 390 |
| TLR10 | 1182 | 1725 | 428 |
| CSF3 | 1183 | 1726 | 391 |
| JUN | 1184 | 1727 | 409 |
| CLEC7A | 1185 | 1728 | 421 |
| AVIL | 1186 | 1729 | 403 |
| IL4 | 1187 | 1730 | 392 |
| GHR | 1188 | 1731 | 437 |
| REG3A | 1189 | 1732 | 396 |
| TNFSF4 | 1190 | 1733 | 428 |
| TPH1 | 1191 | 1734 | 439 |
| HNRNPA1 | 1192 | 1735 | 420 |
| XCL1 | 1193 | 1736 | 433 |
| IL7 | 1194 | 1737 | 422 |
| CD40LG | 1195 | 1738 | 395 |
| TAGAP | 1196 | 1739 | 398 |
| ITLN1 | 1197 | 1740 | 404 |
| MPO | 1198 | 1741 | 442 |
| CCL16 | 1199 | 1742 | 439 |
| NOD2 | 1200 | 1743 | 430 |
| RGMB | 1201 | 1744 | 431 |
| F9 | 1202 | 1745 | 437 |
| TSLP | 1203 | 1746 | 448 |
| TFF3 | 1204 | 1747 | 410 |
| TNFSF8 | 1205 | 1748 | 405 |
| NR1D2 | 1206 | 1749 | 398 |
| PTGER4 | 1207 | 1750 | 426 |
| EPO | 1208 | 1751 | 422 |
| ST8SIA1 | 1209 | 1752 | 390 |
| PDGFA | 1210 | 1753 | 441 |
| CCL2 | 1211 | 1754 | 393 |
| THPO | 1212 | 1755 | 439 |
| BMP2 | 1213 | 1756 | 438 |
| RELT | 1214 | 1757 | 418 |
| PPBP | 1215 | 1758 | 399 |
| CD4 | 1216 | 1759 | 406 |
| SRC | 1217 | 1760 | 391 |
| CSF2RB | 1218 | 1761 | 410 |
| FOXP3 | 1219 | 1762 | 426 |
| IL23R | 1220 | 1763 | 414 |
| ZNF365 | 1221 | 1764 | 390 |
| DMBT1 | 1222 | 1765 | 423 |
| CSF3R | 1223 | 1766 | 401 |
| TLR3 | 1224 | 1767 | 436 |
| CHN1 | 1225 | 1768 | 393 |
| IL1R1 | 1226 | 1769 | 428 |
| CCR3 | 1227 | 1770 | 442 |
| INHBC | 1228 | 1771 | 419 |
| PI3 | 1229 | 1772 | 393 |
| CDKN2A | 1230 | 1773 | 441 |
| IL15RA | 1231 | 1774 | 404 |
| SATB2 | 1232 | 1775 | 413 |
| IL24 | 1233 | 1776 | 426 |
| TNFRSF1B | 1234 | 1777 | 414 |
| SLC25A20 | 1235 | 1778 | 443 |
| MOCOS | 1236 | 1779 | 401 |
| CCDC122 | 1237 | 1780 | 405 |
| CSF2RA | 1238 | 1781 | 400 |
| FCGBP | 1239 | 1782 | 442 |
| SMOC2 | 1240 | 1783 | 420 |
| TNFRSF11A | 1241 | 1784 | 396 |
| S1PR5 | 1242 | 1785 | 429 |
| FASLG | 1243 | 1786 | 443 |
| CCL26 | 1244 | 1787 | 424 |
| CCL4 | 1245 | 1788 | 390 |
| IL1A | 1246 | 1789 | 435 |
| SLC15A1 | 1247 | 1790 | 450 |
| DEFB4A | 1248 | 1791 | 402 |
| IL1RAP | 1249 | 1792 | 446 |
| BMPR2 | 1250 | 1793 | 396 |
| POU5F1 | 1251 | 1794 | 430 |
| TNFRSF8 | 1252 | 1795 | 409 |
| BEST4 | 1253 | 1796 | 418 |
| PLEKHO2 | 1254 | 1797 | 394 |
| PPIA | 1255 | 1798 | 425 |
| TNFSF10 | 1256 | 1799 | 423 |
| CCL21 | 1257 | 1800 | 390 |
| CXCL8 | 1258 | 1801 | 399 |
| GUCA2A | 1259 | 1802 | 399 |
| TNFRSF13B | 1260 | 1803 | 391 |
| RGS3 | 1261 | 1804 | 390 |
| IL13RA1 | 1262 | 1805 | 411 |
| TBP | 1263 | 1806 | 448 |
| CXCR4 | 1264 | 1807 | 408 |
| IL21R | 1265 | 1808 | 428 |
| TNFRSF4 | 1266 | 1809 | 391 |
| PF4 | 1267 | 1810 | 444 |
| TNF | 1268 | 1811 | 419 |
| AMH | 1269 | 1812 | 392 |
| CA7 | 1270 | 1813 | 449 |
| FABP1 | 1271 | 1814 | 433 |
| CCR2 | 1272 | 1815 | 425 |
| CXCR3 | 1273 | 1816 | 415 |
| RORA | 1274 | 1817 | 396 |
| MAPK3 | 1275 | 1818 | 444 |
| PIGR | 1276 | 1819 | 436 |
| IL6 | 1277 | 1820 | 415 |
| ZAP70 | 1278 | 1821 | 426 |
| LCN2 | 1279 | 1822 | 419 |
| DRAIC | 1280 | 1823 | 404 |
| ICAM1 | 1281 | 1824 | 432 |
| IL17F | 1282 | 1825 | 407 |
| TNFSF13 | 1283 | 1826 | 426 |
| CXCR2 | 1284 | 1827 | 419 |
| SERPINE1 | 1285 | 1828 | 450 |
| CHGB | 1286 | 1829 | 442 |
| IFNGR2 | 1287 | 1830 | 448 |
| CLEC2D | 1288 | 1831 | 402 |
| CXCL9 | 1289 | 1832 | 439 |
| SIGIRR | 1290 | 1833 | 408 |
| TRPM5 | 1291 | 1834 | 427 |
| CXCL1 | 1292 | 1835 | 419 |
| TNFRSF1A | 1293 | 1836 | 405 |
| TNFRSF18 | 1294 | 1837 | 432 |
| ATG4D | 1295 | 1838 | 408 |
| IFNK | 1296 | 1839 | 390 |
| CD28 | 1297 | 1840 | 398 |
| TNFSF11 | 1298 | 1841 | 420 |
| CCL27 | 1299 | 1842 | 390 |
| ITGAL | 1300 | 1843 | 400 |
| AQP8 | 1301 | 1844 | 392 |
| PDGFRA | 1302 | 1845 | 430 |
| BEST2 | 1303 | 1846 | 441 |
| GDF5 | 1304 | 1847 | 434 |
| IL18 | 1305 | 1848 | 432 |
| FLT1 | 1306 | 1849 | 390 |
| CXCL2 | 1307 | 1850 | 434 |
| VEGFB | 1308 | 1851 | 390 |
| IRAG2 | 1309 | 1852 | 391 |
| KCNN4 | 1310 | 1853 | 393 |
| CCL25 | 1311 | 1854 | 437 |
| MTOR | 1312 | 1855 | 433 |
| RPS18 | 1313 | 1856 | 422 |
| IL10 | 1314 | 1857 | 423 |
| HMGB1 | 1315 | 1858 | 397 |
| IL17RE | 1316 | 1859 | 412 |
| DLG5 | 1317 | 1860 | 448 |
| FLT3LG | 1318 | 1861 | 402 |
| CCR9 | 1319 | 1862 | 421 |
| HLA-DRA | 1320 | 1863 | 392 |
| IL25 | 1321 | 1864 | 426 |
| IL1R2 | 1322 | 1865 | 444 |
| RNF31 | 1323 | 1866 | 393 |
| TNFRSF10C | 1324 | 1867 | 390 |
| NGFR | 1325 | 1868 | 405 |
| CXCL11 | 1326 | 1869 | 399 |
| NTS | 1327 | 1870 | 441 |
| IL23A | 1328 | 1871 | 412 |
| IRAK3 | 1329 | 1872 | 443 |
| IRGM | 1330 | 1873 | 390 |
| PRKACB | 1331 | 1874 | 403 |
| PTEN | 1332 | 1875 | 421 |
| CD40 | 1333 | 1876 | 450 |
| PSMD4 | 1334 | 1877 | 402 |
| IL20RA | 1335 | 1878 | 434 |
| BMPR1B | 1336 | 1879 | 410 |
| TNFSF14 | 1337 | 1880 | 423 |
| NLRP6 | 1338 | 1881 | 434 |
| TGFBR3 | 1339 | 1882 | 409 |
| OSM | 1340 | 1883 | 431 |
| NFKB1 | 1341 | 1884 | 392 |
| FASN | 1342 | 1885 | 408 |
| IL22RA2 | 1343 | 1886 | 424 |
| TAC1 | 1344 | 1887 | 401 |
| GCG | 1345 | 1888 | 440 |
| FLT4 | 1346 | 1889 | 404 |
| TNFSF18 | 1347 | 1890 | 417 |
| IL18R1 | 1348 | 1891 | 393 |
| XCR1 | 1349 | 1892 | 413 |
| PDCD1 | 1350 | 1893 | 446 |
| DUOX2 | 1351 | 1894 | 397 |
| IL21 | 1352 | 1895 | 447 |
| SST | 1353 | 1896 | 415 |
| MME | 1354 | 1897 | 444 |
| CCL23 | 1355 | 1898 | 416 |
| TNFRSF11B | 1356 | 1899 | 397 |
| CUL2 | 1357 | 1900 | 410 |
| ACVR2B | 1358 | 1901 | 399 |
| MET | 1359 | 1902 | 442 |
| HORMAD2 | 1360 | 1903 | 439 |
| RIPK3 | 1361 | 1904 | 444 |
| TLR7 | 1362 | 1905 | 402 |
| TNFRSF6B | 1363 | 1906 | 399 |
| CD101 | 1364 | 1907 | 422 |
| S100A9 | 1365 | 1908 | 391 |
| NLRP7 | 1366 | 1909 | 393 |
| CCL8 | 1367 | 1910 | 437 |
| CCR6 | 1368 | 1911 | 422 |
| CA2 | 1369 | 1912 | 417 |
| GHRL | 1370 | 1913 | 415 |
| TYK2 | 1371 | 1914 | 431 |
| CASP3 | 1372 | 1915 | 432 |
| INHBB | 1373 | 1916 | 407 |
| TNFRSF21 | 1374 | 1917 | 432 |
| PDGFRB | 1375 | 1918 | 440 |
| CXCL16 | 1376 | 1919 | 390 |
| TOLLIP | 1377 | 1920 | 417 |
| PRLR | 1378 | 1921 | 413 |
| AHR | 1379 | 1922 | 419 |
| NCF4 | 1380 | 1923 | 428 |
| MAP3K8 | 1381 | 1924 | 411 |
| TNFAIP3 | 1382 | 1925 | 448 |
| TNFSF15 | 1383 | 1926 | 447 |
| FUT3 | 1384 | 1927 | 416 |
| BIRC3 | 1385 | 1928 | 450 |
| LIFR | 1386 | 1929 | 391 |
| IKBKG | 1387 | 1930 | 409 |
| VEGFA | 1388 | 1931 | 435 |
| TGFBR2 | 1389 | 1932 | 395 |
| B2M | 1390 | 1933 | 417 |
| TGFB2 | 1391 | 1934 | 394 |
| STAT1 | 1392 | 1935 | 422 |
| PDGFC | 1393 | 1936 | 392 |
| CD14 | 1394 | 1937 | 444 |
| EPCAM | 1395 | 1938 | 417 |
| EDA | 1396 | 1939 | 406 |
| MUC19 | 1397 | 1940 | 413 |
| EGF | 1398 | 1941 | 438 |
| OLFM4 | 1399 | 1942 | 421 |
| TIRAP | 1400 | 1943 | 423 |
| MST1 | 1401 | 1944 | 421 |
| PPARG | 1402 | 1945 | 444 |
| CCL17 | 1403 | 1946 | 419 |
| FCGR3A | 1404 | 1947 | 441 |
| CCL18 | 1405 | 1948 | 396 |
| TLR6 | 1406 | 1949 | 412 |
| KITLG | 1407 | 1950 | 411 |
| TCF7L2 | 1408 | 1951 | 423 |
| MLN | 1409 | 1952 | 398 |
| CIITA | 1410 | 1953 | 391 |
| IL2RA | 1411 | 1954 | 440 |
| TLR2 | 1412 | 1955 | 450 |
| IL19 | 1413 | 1956 | 392 |
| LRRK2 | 1414 | 1957 | 421 |
| CNTFR | 1415 | 1958 | 433 |
| ULK1 | 1416 | 1959 | 415 |
| IL17RC | 1417 | 1960 | 413 |
| TNFRSF12A | 1418 | 1961 | 427 |
| DEFA6 | 1419 | 1962 | 428 |
| GLI1 | 1420 | 1963 | 411 |
| IL17B | 1421 | 1964 | 400 |
| THY1 | 1422 | 1965 | 422 |
| IL17RB | 1423 | 1966 | 433 |
| PTCH1 | 1424 | 1967 | 394 |
| TNFRSF10D | 1425 | 1968 | 391 |
| CXCL5 | 1426 | 1969 | 427 |
| FGFBP2 | 1427 | 1970 | 434 |
| REG1A | 1428 | 1971 | 420 |
| ICOSLG | 1429 | 1972 | 394 |
| CX3CL1 | 1430 | 1973 | 444 |
| TNFRSF10B | 1431 | 1974 | 404 |
| VEGFD | 1432 | 1975 | 420 |
| IL3RA | 1433 | 1976 | 421 |
| CX3CR1 | 1434 | 1977 | 435 |
| IL20RB | 1435 | 1978 | 437 |
| IL9R | 1436 | 1979 | 411 |
| MYC | 1437 | 1980 | 415 |
| CDKAL1 | 1438 | 1981 | 417 |
| ASCL2 | 1439 | 1982 | 391 |
| PTPN22 | 1440 | 1983 | 417 |
| TNFRSF25 | 1441 | 1984 | 390 |
| APAF1 | 1442 | 1985 | 397 |
| SPDEF | 1443 | 1986 | 427 |
| GAPDH | 1444 | 1987 | 429 |
| ANPEP | 1445 | 1988 | 432 |
| SLC22A4 | 1446 | 1989 | 444 |
| MMP12 | 1447 | 1990 | 450 |
| DAP | 1448 | 1991 | 401 |
| ADAMDEC1 | 1449 | 1992 | 445 |
| SLC15A4 | 1450 | 1993 | 418 |
| IL5RA | 1451 | 1994 | 422 |
| EDAR | 1452 | 1995 | 437 |
| CTLA4 | 1453 | 1996 | 450 |
| CYTOR | 1454 | 1997 | 426 |
| OSMR | 1455 | 1998 | 442 |
| CXCR6 | 1456 | 1999 | 407 |
| IFNLR1 | 1457 | 2000 | 409 |
| IL2RB | 1458 | 2001 | 409 |
| APC | 1459 | 2002 | 406 |
| IFNB1 | 1460 | 2003 | 396 |
| MPL | 1461 | 2004 | 447 |
| LEPR | 1462 | 2005 | 448 |
| CCL1 | 1463 | 2006 | 445 |
| CHGA | 1464 | 2007 | 415 |
| FUT2 | 1465 | 2008 | 407 |
| CXCR1 | 1466 | 2009 | 409 |
| CLDN8 | 1467 | 2010 | 407 |
| IL4R | 1468 | 2011 | 450 |
| IL5 | 1469 | 2012 | 404 |
| CLCA1 | 1470 | 2013 | 438 |
| PDGFB | 1471 | 2014 | 392 |
| TNFRSF17 | 1472 | 2015 | 411 |
| OTOP2 | 1473 | 2016 | 446 |
| PRL | 1474 | 2017 | 419 |
| CBR3-AS1 | 1475 | 2018 | 447 |
| NCF2 | 1476 | 2019 | 429 |
| PGLYRP1 | 1477 | 2020 | 416 |
| TREM1 | 1478 | 2021 | 413 |
| IL10RB | 1479 | 2022 | 436 |
| CYLD | 1480 | 2023 | 443 |
| BCAR1 | 1481 | 2024 | 433 |
| ACVR1 | 1482 | 2025 | 426 |
| GIP | 1483 | 2026 | 433 |
| CCL3 | 1484 | 2027 | 404 |
| NLRP3 | 1485 | 2028 | 423 |
| CCL24 | 1486 | 2029 | 428 |
| JAK2 | 1487 | 2030 | 432 |
| IRF8 | 1488 | 2031 | 437 |
| CCR5 | 1489 | 2032 | 410 |
| PGK1 | 1490 | 2033 | 429 |
| SLC26A2 | 1491 | 2034 | 447 |
| ATG7 | 1492 | 2035 | 417 |
| FFAR2 | 1493 | 2036 | 434 |
| IFI44 | 1494 | 2037 | 394 |
| NR1H2 | 1495 | 2038 | 417 |
| PSTPIP1 | 1496 | 2039 | 403 |
| FFAR3 | 1497 | 2040 | 449 |
| MKI67 | 1498 | 2041 | 399 |
| HGF | 1499 | 2042 | 447 |
| WFDC2 | 1500 | 2043 | 396 |
| DEFB1 | 1501 | 2044 | 393 |
| CSF1R | 1502 | 2045 | 445 |
| AMHR2 | 1503 | 2046 | 416 |
| LEP | 1504 | 2047 | 419 |
| PF4V1 | 1505 | 2048 | 426 |
| CCR4 | 1506 | 2049 | 450 |
| NLRP10 | 1507 | 2050 | 440 |
| CD86 | 1508 | 2051 | 406 |
| IL12RB2 | 1509 | 2052 | 443 |
| CSF1 | 1510 | 2053 | 390 |
| IL26 | 1511 | 2054 | 401 |
| PTPN2 | 1512 | 2055 | 436 |
| CD68 | 1513 | 2056 | 391 |
| IFI44L | 1514 | 2057 | 447 |
| INHBA | 1515 | 2058 | 433 |
| AGR2 | 1516 | 2059 | 407 |
| CD44 | 1517 | 2060 | 438 |
| KLF6 | 1518 | 2061 | 411 |
| IGF1 | 1519 | 2062 | 396 |
| EPOR | 1520 | 2063 | 427 |
| INS | 1521 | 2064 | 450 |
| CCL22 | 1522 | 2065 | 425 |
| TLR9 | 1523 | 2066 | 402 |
| IL12B | 1524 | 2067 | 437 |
| HAPLN1 | 1525 | 2068 | 398 |
| BIRC2 | 1526 | 2069 | 420 |
| KIT | 1527 | 2070 | 437 |
| TNFRSF9 | 1528 | 2071 | 434 |
| IFNL1 | 1529 | 2072 | 424 |
| CCL7 | 1530 | 2073 | 390 |
| GUSB | 1531 | 2074 | 415 |
| ADIPOQ | 1532 | 2075 | 401 |
| EGFR | 1533 | 2076 | 398 |
| ATG5 | 1534 | 2077 | 416 |
| NLRP1 | 1535 | 2078 | 441 |
| TNFSF12 | 1536 | 2079 | 410 |
| CCR10 | 1537 | 2080 | 401 |
| CD2 | 1538 | 2081 | 443 |
| NELL1 | 1539 | 2082 | 416 |
| IL6R | 1540 | 2083 | 443 |
| VEGFC | 1541 | 2084 | 405 |
| IL17A | 1542 | 2085 | 395 |
| TFRC | 1543 | 2086 | 437 |
| IFNAR1 | 1544 | 2087 | 430 |
| CCL11 | 1545 | 2088 | 447 |
| NOX1 | 1546 | 2089 | 427 |
| CTF1 | 1547 | 2090 | 424 |
| FAS | 1548 | 2091 | 403 |
| IL12RB1 | 1549 | 2092 | 407 |
| IL2 | 1550 | 2093 | 395 |
| IL36A | 1551 | 2094 | 397 |
| MIF | 1552 | 2095 | 450 |
| SEL1L | 1553 | 2096 | 434 |
| TLR4 | 1554 | 2097 | 416 |
| IL1B | 1555 | 2098 | 432 |
| C1GALT1C1 | 1556 | 2099 | 398 |
| ABCA1 | 1557 | 2100 | 441 |
| CXCR5 | 1558 | 2101 | 407 |
| IL17D | 1559 | 2102 | 414 |
| KDR | 1560 | 2103 | 432 |
| IL22 | 1561 | 2104 | 436 |
| LTBR | 1562 | 2105 | 406 |
| CCR8 | 1563 | 2106 | 406 |
| TNFSF9 | 1564 | 2107 | 445 |
| CXCL3 | 1565 | 2108 | 393 |
| IL12A | 1566 | 2109 | 448 |
| FLT3 | 1567 | 2110 | 406 |
| IL13RA2 | 1568 | 2111 | 405 |
| CXCL14 | 1569 | 2112 | 398 |
| TLR8 | 1570 | 2113 | 406 |
| CCK | 1571 | 2114 | 421 |
| IL20 | 1572 | 2115 | 449 |
| CXCL10 | 1573 | 2116 | 392 |
| TNFRSF14 | 1574 | 2117 | 424 |
| RNF186 | 1575 | 2118 | 449 |
| YWHAZ | 1576 | 2119 | 444 |
| KLK3 | 1577 | 2120 | 407 |
| DNMT3B | 1578 | 2121 | 441 |
| CARD9 | 1579 | 2122 | 426 |
| ATG16L1 | 1580 | 2123 | 402 |
| INHBE | 1581 | 2124 | 429 |
| TNFSF13B | 1582 | 2125 | 430 |
| HDAC6 | 1583 | 2126 | 439 |
| CEACAM6 | 1584 | 2127 | 426 |
| TOP2A | 1585 | 2128 | 391 |
| IHH | 1586 | 2129 | 405 |
| IL15 | 1587 | 2130 | 435 |
| IRF1 | 1588 | 2131 | 415 |
| IRF5 | 1589 | 2132 | 419 |
| ACKR3 | 1590 | 2133 | 417 |
| PCK1 | 1591 | 2134 | 426 |
| IFNE | 1592 | 2135 | 405 |
| CRLF2 | 1593 | 2136 | 430 |
| CCL15 | 1594 | 2137 | 402 |
| BTNL2 | 1595 | 2138 | 441 |
| NOS2 | 1596 | 2139 | 403 |
| IL2RG | 1597 | 2140 | 394 |
| ERBIN | 1598 | 2141 | 401 |
| TGFB3 | 1599 | 2142 | 401 |
| SLC2A14 | 1600 | 2143 | 406 |
| ITGB2 | 1601 | 2144 | 426 |
| MUC1 | 1602 | 2145 | 423 |
| ABCA8 | 1603 | 2146 | 419 |
| CDKN1A | 1604 | 2147 | 437 |
| CXCL13 | 1605 | 2148 | 391 |
| KLK15 | 1606 | 2149 | 392 |
| CCL28 | 1607 | 2150 | 404 |
| CXCL6 | 1608 | 2151 | 425 |
| LAMB1 | 1609 | 2152 | 405 |
| IFNA1 | 1610 | 2153 | 449 |
| XIAP | 1611 | 2154 | 438 |

1. A method for transcriptome profiling, comprising:
amplifying one or more target RNA sequences from a sample comprising RNA extracted from a fecal sample from a subject to produce amplicons of less than about 500 bases in length; and
sequencing the amplicons.

2. The method of claim 1, wherein the RNA extracted from the fecal sample comprises RNA derived from subject cells and RNA derived from gut bacteria.

3. The method of claim 1, wherein the one or more target RNA sequences are derived from one or more subject genes.

4. The method of claim 3, wherein the one or more subject genes comprise a housekeeping gene, a tissue-specific gene, a cell type-specific gene, a disease related gene, a cell-signaling gene, or combinations thereof.

5. The method of claim 3, further comprising determining gene expression for the one or more subject genes.

6. The method of claim 1, wherein the one or more target RNA sequences are about 300 to about 400 nucleotides in length.

7. The method of claim 1, wherein the amplicons are about 350 to about 500 bases in length and the methods optionally further comprise purifying the amplicons based on size prior to sequencing.

8. The method of claim 1, wherein the amplifying comprises:
contacting the sample with a reverse transcriptase and random hexamer primers under conditions for DNA synthesis to form an cDNA mixture; and
contacting the cDNA mixture with a DNA polymerase and a pair of oligonucleotide primers configured to specifically amplify each of one or more target sequences under conditions for amplicon production.

9. The method of claim 8, wherein the amplicon production is limited cycle PCR amplification.

10. The method of claim 8, wherein each of the oligonucleotide primers comprises an amplicon identifier sequence and each amplicon comprises two amplicon identifier sequences flanking a target sequence.

11. The method of claim 1, further comprising one or both of: removing residual RNA from the cDNA mixture and removing single stranded nucleic acid impurities from the amplicons.

12. A method for diagnosing a disease or disorder in a subject, comprising:
generating a transcriptome profile of subject cells in a fecal sample from the subject by the method of claim 1; and
comparing the transcriptome profile to a healthy control to determine whether the individual has or has an increased likelihood of having the disease or disorder.

13. A method for monitoring the progression or regression of a disease or disorder in a subject, comprising:
acquiring two or more fecal samples from the subject, wherein the two or more fecal samples are separated by a period of time;
generating a transcriptome profile of subject cells in the two or more fecal samples by the method of claim 1;
identifying changes in the transcriptome profile between any of the fecal samples; and
optionally, associating changes in the transcriptome profile with progression or regression of the disease or disorder.

14. The method of claim 12, wherein the disease or disorder is irritable bowel syndrome (IBS), inflammatory bowel diseases (IBD), Crohn's disease (CD), Celiac's disease (CeD), ulcerative colitis (UC), and colon cancer.

15. A method for evaluating gut health in a subject, comprising:
generating a transcriptome profile of subject cells in one or more fecal samples from the subject by the method of claim 1; and
comparing the transcriptome profile of the one or more fecal samples to one or more controls to determine measure of overall gut health;
optionally identifying changes in the transcriptome profile between any of the one or more fecal samples; and associating changes in the transcriptome profile with changes in gut health; and
optionally providing an assessment of gut health.

16. The method of claim 13, wherein the disease or disorder is irritable bowel syndrome (IBS), inflammatory bowel diseases (IBD), Crohn's disease (CD), Celiac's disease (CeD), ulcerative colitis (UC), and colon cancer.
